(19) United States
(12) Patent Application Publication
(10) Pub. No.: US 2001/0053517 A1
(43) Pub. Date: Dec. 20, 2001

(54) DETERMINING NUCLEIC ACID SEQUENCES IN A BIOLOGICAL SAMPLE

(76) Inventors: PETER A. ANTON, LOS ANGELES, CA (US); IAN MCGOWEN, SAN FRANCISCO, CA (US); IRVIN S.SY. CHEN, LOS ANGELES, CA (US); JULIE E. ELLIOTT, SIGNAL HILL, CA (US)

Correspondence Address: ROBERT M. BEDGOOD, PILLSBURY WINTHROP, LLP, 50 FREMONT STREET, SAN FRANCISCO, CA, 94105, US

(21) Appl. No.: 09/344,325

(22) Filed: Jun. 24, 1999

(51) Int. Cl. C12Q 1/68

(52) U.S. Cl. 435/6

(57) ABSTRACT

Provided are methods for determining the presence of a specific nucleic acid sequence in a non-fluid biological sample. Also provided are methods for detecting and determining the whole body viral burden of a virus in a subject.

### TECHNICAL FIELD

This invention relates to methods and compositions used to assay a nucleic acid burden in tissue or other body compartments. Specifically the invention concerns the determination of HIV viral infections by determining viral burden.

### BACKGROUND

The HIV infection cycle begins with the entry of the virus into the target cell. The human CD4 is believed to be the primary receptor on T cells recognized by HIV. The binding of the HIV envelope glycoprotein (env) to the CD4 receptor results in the fusion of virus and cell membranes, which in turn facilitates virus entry into the host. The eventual expression of env on the surface of the HIV-infected host cell enables this cell to fuse with uninfected CD4-positive cells, thereby spreading the virus. However, HIV can also enter other cells such as monocytes, B cells, and dendritic cells, which can serve as viral reservoirs, even though they may not express CD4. Cytokines are known to affect HIV replication. Pro-inflammatory cytokines promote HIV replication (Fauci, Nature 384:529-534, 1996), while β-chemokines inhibit the replication of obligate CCR5 utilizing viruses (Moore, et al., J Virol. 70:551-562, 1996), and enhance the replication of CXCR4 utilizing viral isolates (Dolei, et al., AIDS 12:183-190,1998).

The normal intestinal tract is characterized by a low level of mild inflammation, which is fueled by constitutive levels of locally secreted chemokines and cytokines (Shanahan and Anton, Gut Peptides, J. Walsh eds. (Raven Press, Ltd, New York, 1994, page 851; Schreiber et al., Gastroenterology 101:1020 (1991); MacDermott et al., Inflammatory Bowel Diseases 4, 54 (1998); Luster, N.Engl. J Med. 338:436 (1998)). In healthy controls, gastrointestinal lymphocytes are known to differ functionally and phenotypically from their peripheral blood counterparts (Allison et al., Gastroenterology 99:421 (1990); Jarry et al., Eur. J Immunol. 20:1097 (1990); McGowan et al., Neuroimmunomodulation 4:70 (1997)). Virtually all mucosal CD4+ lymphocytes express activation markers and are of the CD45RO+ memory subset (Schieferdecker et al., J Virol. 149:2816 (1992)). In the setting of HIV−1 infection, various phenotypic abnormalities of gut T lymphocytes have been described often associated with depletion of CD4+ lymphocytes (Schnieder et al., Clin. Exp. Immunol. 95:430 (1994)).

Quantitative measurement of HIV RNA viral titers in plasma has quickly become the mainstay of clinical management and the primary index of therapeutic efficacy in clinical trials of combination therapies for HIV. Reducing plasma viral load to an increasingly lowered threshold of detectability has been the clinical target the sensitivity of which is increasingly refined. Awareness of the persistence of viral activity in tissue (i.e., lymph nodes, the initial site of the observation) and/or blood cells in subjects with prolonged periods of undetectable levels of HIV in the plasma has fueled interest in the development of techniques that are as effective as the plasma viral load kits commercially available for monitoring viral activity.

Reduction in plasma viral load indicates a reduced rate of viral replication. However, the extent of viral replication throughout a subject's body is a crucial variable that cannot be inferred directly from the plasma viral load.

### SUMMARY

The present invention is based upon the discovery that pathogens, such as viruses (e.g., HIV and SIV) and bacteria can become established in tissue compartments and immune cells deep within various tissues of the body shortly after infection. These deep tissues or compartments reflect the greatest percentage of total body pathogen burden. For example, the deep tissues or compartments reflect the greatest percentage of total body viral burden, and particularly the greatest percentage of HIV viral burden. As such, the currently available tests of viral burden only measure the concentration of virus in the patient's blood. One crucial limitation of these measurements is that even when virus is not detectable in blood, there is still a substantial viral burden and often ongoing replication in the patient's tissues.

Additionally, the present invention provides methods, compositions and kits for maintaining and determining the quantity of a specific nucleic acids in a sample. Such methods utilize a “spiking” techniques in which the biological sample is spiked with an internal standard of a known quantity at the time of obtaining the biological sample in order to determine the natural degradation of the sample over time, such as during shipment.

In one embodiment the present invention provides a method of determining a specific nucleic acid sequence in a non-fluid biological sample, by obtaining a biolgical sample from the subject and quantifying the amount of the specific nucleic acid present in the sample. The specific nucleic acid can be related to a pathologic condition, such as a viral or bacterial infection. The sample can selected from the group consisting of mucosal tissue, skin tissue, lymph tissue, ocular tissue, pulmonary tissue, and liver tissue.

In another embodiment, the present invention provides, a method for determining the effect of a therapeutic treatment on whole body viral load in a subject, comprising determining the viral load in the plasma of the subject and the viral load in a tissue-biopsy of the subject compared to a standard whole body viral load, wherein a change in the viral load in the blood and the tissue is indicative of an effect.

In yet another embodiment, the present invention provides a kit for obtaining a non-fluid biological sample from the skin, the kit having a collection device a known quantity of a nucleic acid; and a cell lysis buffer suitable of preserving nucleic acids.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows pre-amplification handling of tissue biopsy samples results in a 5-10% RNA loss. Sero-negative samples were ‘spiked’ with 250 copies of the standard LTR sequence pre-extraction (left) and in a parallel sample post-extraction (right). Digitized quantification of ³²P emission demonstrated a 5-10% difference between pre and post extraction additions of the same amount of LTR RNA. Standards demonstrate an assay sensitivity of 10 copies.

FIG. 2 shows the ability to detect and quantify levels of HIV RNA in rectosigmoid biopsies from 4 patients with variable but detectable plasma viral loads. Samples had 10² to 10³ copies HIV RNA in tissue per μg total RNA added. Quantified values are corrected for percentage RNA loss and therefore reflect in vivo levels. Assay sensitivity is 10 copies.

FIG. 3 demonstrates the reproducibility of the invention in patients with detectable plasma viral load by showing that biopsies from the same subject in different quadrants at the same level in the rectosigmoid yield similar amounts of HIV RNA. Two representative subjects are shown with 3 biopsies (Patient #1) or 2 biopsies (Patient #2) taken at 30 cm and run in duplicate. For each subject, there was a 0.2 log SD between samples.

FIG. 4 shows that HIV RNA can be successfully extracted from subject's tissue (rectosigmoid biopsies) who have UNdetectable plasma viral load (less than 400 copies/ml) in an assay with a sensitivity of 10 copies per reaction. Results can be expressed as actual copy numbers per reaction or standardized as number of copies per biopsy.

FIG. 5 shows that HIV RNA can be reproducibly quantified from different biopsies at the same level in the rectosigmoid colon in patients with undetectable viral load in the plasma (<400 copies per ml). These results in 15 subjects demonstrate HIV RNA yield over a 3 log₁₀ range with a sensitivity of 10 copies per reaction and an inter-sample variation of less than 0.2 log₁₀ copies per g tissue RNA. These results confirm there is anatomical variation in tissue HIV RNA within the same 30 cm level in the rectosigmoid colon.

FIG. 6 shows that HIV DNA can also be reliably quantitated from tissue biopsies acquired from patients with undetectable plasma viral load. Representative samples from 4 subjects are shown with standard curve for HIV LTR to detect HIV DNA by direct PCR with a sensitivity of 10 copies per reaction. Samples were standardized with an internal control (housekeeping gene), P-globin. Results are reported in both actual copy numbers per reaction and calculated as number of copies per 2×10⁶ β-globin copies (or 1×106 cells).

FIG. 7 is a tabular presentation of results of 19 patients studied with undetectable plasma viral load (<400 copies per ml) for >3 months who underwent tissue acquisition to determine tissue levels of HIV activity as expressed by both HIV RNA and HIV DNA. This demonstrates the utility of using a single biopsy to quantify both HIV RNA and DNA with the aim to establish an index to guide future therapy. There is a 3-log₁₀ range in results for HIV RNA. 88% of subjects retain detectable levels of tissue RNA despite persistent undetectable levels in plasma. 100% of subjects have detectable HIV DNA.

FIG. 8a and 8b show the ability to reliably extract and quantitate HIV RNA and HIV DNA from tissue samples from subjects with undetectable plasma viral load using the ‘ultrasensitive’ assay (<40 copies/ml plasma). The results show that HIV RNA is detected in 67% of subject's tissue samples when undetectable in plasma (8a) and HIV DNA is found in 100% (8b).

FIG. 9 shows novel clinical applicability of this invention used in a study of gene therapy for treating HIV infection. Five subjects with detectable plasma viral HIV RNA were enrolled and received protocol-driven re-infusion of their own apheresed and genetically altered cells. Impact on plasma and tissue viral burden was assessed at baseline, days 3, 7, and 14. Results show a trend toward decreasing tissue viral burden when no changes were appreciated in plasma. This contributed to the design of a multicentered trial of gene-therapy for HIV-infected subjects with undetectable plasma viral load (<40 copies/ml for >3 months) for which change in tissue viral burden using this invention were the primary endpoints.

FIG. 10a, b and c show novel clinical applicability of this invention to use tissue samples to quantify viral burden and subsequently determine patterns of genotypic and phenotypic viral resistance using the same biopsy samples. Importantly, because resistance testing requires at least 1.2 kb intact sequences and grow the viruses for phenotypic testing, these results demonstrate that the HIV detected is replication-competent HIV RNA, not simply short fragments of HIV RNA. FIG. ure 10a shows the clinical features of the subjects studied including present medications to which viral resistance had developed as evidenced by incompletely suppressed, break-through HIV RNA in plasma. Tissue HIV RNA levels are detectable in these subjects as inferred from FIG. 4 and 5. FIG. 10b shows the patterns of genotypic and phenotypic resistance in contemporaneous plasma and the tissue (gut) samples from these subjects with detectable plasma viral load. There is a high concordance in sensitivity and resistance profiles to 14 antiviral medications. FIG. 10c shows tabular results of drug-resistance patterns in patients with undetectable plasma viral load. These results are in the process of being finalized but demonstrate the ability to use tissue samples to first quantity the amount of virus and subsequently determine drug resistance patterns in this population of patients. This information will provide logical and scientific guidance for intensifying therapy to further decrease total body viral burden in those without detectable plasma levels.

FIG. 11 shows the incorporated ability to trace recoverability of RNA from the point of acquisition during the patient's procedure. By including an internal control separate from the tissue sample, one is able to trace the loss/recovery ratios throughout the extraction and amplification process and subsequently correct detected amounts for process-related degradation. These results show gel and tabulated data from 8 subjects sero-negative for HIV (and therefore without HIV LTR sequence in their tissue) with known amounts of HIV LTR added to the frozen sample pre-assay. Recovery was nearly 100% regardless of whether the sample was fully or partially utilized for RNA quantitation. For HIV-positive subjects, similar experiments have been performed using cyclophilin RNA as internal control; a variety of non-human RNA sequences can be utilized to provide the same results.

FIG. 12 shows similar recoverability and capacity to target loss of DNA during acquisition, extraction and amplification. Results in gel and tabular form from 2 subjects (2 biopsies each) demonstrate the expected and actual recovery of an added, non-human (firefly luciferase DNA) gene product to tissue samples (rectal biopsies). Recovery regardless of whether the sample was fully utilized or divided (with part later used for RNA quantitation) resulted in >90% recovery. As β-globin is usually run in parallel, these percentages can be used to quantify yield per corrected number of cells.

FIG. 13 shows CCR5 receptor expression on CD4+ lymphocytes from blood and from gut mucosa. Flow cytometry scatter plots (A, B) demonstrate lymphocyte subset analysis to quantify percentages of cells expressing CCR5 and/or CD4 in a representative subject for blood (A) and gut (B). The number on the upper right quadrant of each plot indicates the percentage of CD4+ lymphocytes in that subject that expressed CCR5. (C) The individual data points for the six subjects; data from the blood and gut of each subject. The gut samples of all six subjects had a greater percentage of CCR5 + CD4+ cells compared with the blood (P=0.03); differences range from 2.0-5.4-fold.

FIG. 14 shows the number of CCR5 receptors per cell on CD4+ lymphocytes from blood and from gut mucosa. Flow cytometry histograms (A,B) demonstrate quantitation of CCR5 expression on CD4+ lymphocytes of one of the six subjects for blood (A) and gut (B). The rightward shift of mean fluorescence index in the CCR5+ CD4+ mucosal cells illustrates the increased numbers of CCR5 receptors per CD4+ lymphocyte. The number above the bars in A and B indicates the number of molecules of CCR5 expressed per CCR5+ CD4+ lymphocyte in the blood and gut of that individual. (C) The gut samples of all six subjects had higher expression of CCR5 compared with the blood (P=.03); differences range from 1.4 to 3.5-fold. Symbols for each person are the same as those used in FIG. 13.

FIG. 15 shows the number of picograms of p24 produced by MMC and PBMC after infection with HIV_{SX} or HIV_{NL}4-3. Line graphs indicate the p24 production (picograms of p24 per 10⁴ CD4+ lymphocytes) at 18, 72, and 130 hours after a 3-hour infection with either M-tropic HIV_{SX} (A) or T-tropic HIV_{NL}4-3 (B). After 72 and 130 hours the supernatants from the MMCs cultured in the presence of 20 IU/mL of IL-2 (A) contained greater concentrations of p24 than the supernatants from either PBMC grown with (∘) or without (▾) 20 IU/mL of IL-2. The greater p24 production from the cultured mucosal cells suggests that they are more susceptible than PBMC to replication of M-, or T-tropic HIV−1.

FIG. 16 shows the 3 day/IL-2 culture for isolation of mucosal mononuclear cells yields increased numbers of CD45+,CD3+ CD4+ and CD8+ cells as compared to conventional collagenase/dispase digestion. The mononuclear cell populations isolated by each technique do not appear to differ significantly in their T cell subset make-up.

FIG. 17 shows that the isolation process does not alter relevant receptor expression. Flow diagrams of peripheral blood mononuclear cells (PBMC) stained directly [upper panels] with antibodies to CD4, CD8, CCR5 or CXCR4 as identified on the horizontal and vertical axes. Lower panels show results of parallel staining of the same individual's PBMC following exposure to the isolation process used for mucosal mononuclear cells.

FIG. 18 shows increased mucosal compared to blood CCR5 expression on CD4+ T cells are detected in normal, inflammatory and HIV-infected samples. Mean percentages of CD4+ and CCR5+ double-stained cells are shown from seronegative healthy controls (n=6), inflammatory controls (n=4) and HIV-infected (n=8). P values under the subjects category on the x-axis identify the significance between blood and gut cells within the clinical group. P values at the top of the graph identify significance levels between the CCR5-expressing CD4 T cells in the mucosal compartment between clinical groups.

FIG. 19 shows that CCR5+ CD4:CD8 ratios in blood and gut decline in IBD and HIV. The left panel shows relative CCR5 expression in blood from healthy, sero-negative controls, seronegative inflammatory controls and subjects with stable HIV infection. The changing ratios of CCR5-expressing CD4+ T cells to CCR5-expressing CD8+ T cells are boxed underneath. Similar presentations for mucosal lymphocytes are shown on the right.

### DETAILED DESCRIPTION

It must be noted that as used herein and in the appended claims, the singular forms “a”, “and”, and “the” include the plural unless the context clearly dictates otherwise. Thus, for example, reference to “a cell” includes a plurality of such cells. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the cell lines, antibodies, and methodologies which are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The present invention provides a method for determining the nucleic acid burden (e.g., the viral burden) in a subject. Such methods detect, for example, the presence or absence of viral nucleic acids. For example, quantitiating tissue levels of specific RNA and/or DNA sequences that accurately reflect in vivo status. More particularly, the present invention utilizes both RNA and DNA to detect viral burden in tissue compartments. The invention provides a method for determining viral burden in a subject wherein the subject does not present with plasma viral load. Such methods are advantageous in determining the best therapeutic route or treatment for modulating or further reducing the viral load in a subject or for confirming new infection. Furthermore, the methods of the invention are useful for determining the best therapy for affecting viral load in a subject.

The present invention is applicable to the detectin of specific nucleic acid seequences shown to be associated with hujan pathology including oncogenic or heritable disorders as well as infections from a variety of organisms, including, for example, bacterial infections, viral infections (e.g., retroviral infection such as HIV and SIV) as well as other type of infections known to those of skill in the art. For example, retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The family Retroviridae are enveloped single-stranded RNA viruses typically infect mammals, such as, for example, bovines, monkeys, sheep, and humans. Retroviruses are unique among RNA viruses in that their multiplication involves the synthesis of a DNA copy of the RNA which is then integrated into the genome of the infected cell.

The Retroviridae family consists of three groups: the spumaviruses (or foamy viruses) such as the human foamy virus (HFV); the lentiviruses, as well as visna virus of sheep; and the oncoviruses (although not all viruses within this group are oncogenic). The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2) and simian immunodeficiency virus (SIV). In the absence of effective therapy, most individuals infected with a human immunodeficiency virus develop acquired immune deficiency syndrome (AIDS) and succumb to either opportunistic infections and malignancies resulting from either the deterioration of the immune system or the direct effects of the virus. The oncoviruses are further subdivided into groups A, B, C and D on the basis of particle morphology, as seen under the electron microscope during viral maturation. A-type particles represent the immature particles of the B- and D-type viruses seen in the cytoplasm of infected cells. These particles are not infectious. B-type particles bud as mature virions from the plasma membrane by the enveloping of intracytoplasmic A-type particles. At the membrane they possess a toroidal core of −75 nm, from which long glycoprotein spikes project. After budding, B-type particles contain an eccentrically located, electron-dense core. The prototype B-type virus is mouse mammary tumor virus (MMTV). No intracytoplasmic particles can be observed in cells infected by C-type viruses. Instead, mature particles bud directly from the cell surface via a crescent ‘C’-shaped condensation which then closes on itself and is enclosed by the plasma membrane. Envelope glycoprotein spikes may be visible, along with a uniformly electron-dense core. Budding may occur from the surface plasma membrane or directly into intracellular vacuoles. The C-type viruses are the most commonly studied and include many of the avian and murine leukemia viruses. Bovine leukemia virus (BLV), and the human T-cell leukemia viruses types I and II (HTLV-I/II) are similarly classified as C-type particles because of the morphology of their budding from the cell surface. However, they also have a regular hexagonal morphology and more complex genome structures than the prototypic C-type viruses such as the murine leukemia viruses (MLV). D-type particles resemble B-type particles in that they show as ring-like structures in the infected cell cytoplasm, which bud from the cell surface, but the virions incorporate short surface glycoprotein spikes. The electron-dense cores are also eccentrically located within the particles. Mason Pfizer monkey virus (MPMV) is the prototype D-type virus.

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious virions.

The retrovius particle is composed of two identical RNA molecules. Each genome is a positive sense, single-stranded RNA molecule, which is capped at the 5 end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope proteins. The env proteins bind to the cellular receptor for the virus and the particle typically enters the host cell via receptor-mediated endocytosis and/or membrane fusion.

After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome.

The double-stranded linear DNA produced by reverse transcription may, or may not, have to be circularized in the nucleus before integration into the host cell genome. The provirus now has two identical repeats at either end, known as the long terminal repeats (LTR). The junction between the two joined LTR sequences produces the site recognized by a pol product—the integrase protein—which catalyzes integration, such that the provirus is always joined to host DNA two base pairs (bp) from the ends of the LTRs. A duplication of cellular sequences is seen at the ends of both LTRs, reminiscent of the integration pattern of transposable genetic elements. Integration is thought to occur essentially at random within the target cell genome.

Transcription, RNA splicing and translation of the integrated viral DNA is mediated by host cell proteins. Variously spliced transcripts are generated. In the case of the human retroviruses HIV ½ and HTLV-I/II viral proteins are also used to regulate gene expression. The interplay between cellular and viral factors is important in the control of virus latency and the temporal sequence in which viral genes are expressed.

Retroviruses can be transmitted horizontally and vertically. Efficient infectious transmission of retroviruses requires the expression on the target cell of receptors which specifically recognize the viral envelope proteins, although viruses may use receptor-independent, nonspecific routes of entry at low efficiency. In addition, the target cell type must be able to support all stages of the replication cycle after virus has bound and penetrated. Vertical transmission occurs when the viral genome becomes integrated in the germ line of the host. The provirus will then be passed from generation to generation as though it were a cellular gene. Hence endogenous proviruses become established which frequently lie latent, but which can become activated when the host is exposed to appropriate agents.

The oncoviruses (often called the RNA tumor viruses) have been subdivided into two groups of pathogens, namely the acutely transforming and slow transforming retroviruses.

Acutely transforming retroviruses can transform cultured cells and can cause disease rapidly in susceptible animals. These viruses usually carry an oncogene (v-onc) within the viral genome, which is directly responsible for their tumorigenicity, and which is different in each type of virus. The viral oncogenes have been derived from cellular genes that the viruses have acquired, probably as a result of the inclusion of cellular RNA within a viral particle. Subsequent recombination between viral and cellular RNA during reverse transcription leads to the incorporation of the cellular sequences into the viral genome and delivery of this novel unit into the host cell DNA. If the transduced gene normally has a central role in control of cellular growth and differentiation, the changes in coding sequence and/or control of expression that it undergoes on incorporation into the viral genome can render it oncogenic. Such cellular proto-oncogenes (c-onc) may become oncogenic by being placed under novel, virally determined transcriptional control (both quantitatively and temporally), and/or by sustaining critical mutations to the coding sequence. However, full cellular transformation usually requires the expression of v-onc in conjunction with other genetic and epigenetic changes within the target cell.

The slow transforming retroviruses typically do not contain a ‘classical’ oncogene. The mechanism of transformation is believed rather to involve the insertion of provirus near, or in, the coding region of a cellular proto-oncogene, called insertional mutagenesis. The strong promoter and enhancer sequences within the viral LTRs can exert transcriptional effects from distances of up to several kilobase pairs from the proto-oncogene. The normal regulation of expression of the cellular gene is disrupted, and over-expression or inappropriately timed expression can contribute to transformation.

HTLV-1 is a slow-transforming virus, causally associated with adult T-cell leukemia (ATL), but it probably promotes T-cell transformation by a different pathway involving virally encoded regulatory proteins, especially p40tax, which transactivate expression of cellular proto-oncogenes. HIV-1 and 2 have also been implicated in both the direct and indirect promotion of various types of malignancy (such as Kaposi's sarcoma) which present much more frequently in AIDS patients than in the general population. However, the direct role of HIV in malignant transformation remains doubtful as many patients who are immunosuppressed as a result of other infections or treatments (e.g. transplant recipients) also develop tumours at increased rates.

The D-type viruses are not aetiologically associated with malignancy, although MPMV was initially associated with a mammary tumor in a rhesus monkey. D-type viruses cause immune suppression in simian primates but by an unknown mechanism. Immune suppression is also a feature of infection by the lentiviruses (e.g HIV and SIV) and variant strains of feline leukemia virus (FeLV). In infection with HIV and FeLV large amounts of unintegrated proviral DNA have been observed, which may be related to the pathogenesis.

The lentiviruses, including HIV-½ and visna virus of sheep, are associated with slow progressive disease leading to immune suppression and neurological disorders. HIV is the widely recognized causative agent of the acquired immunodeficiency disease syndrome (AIDS). The pharmaceutical compositions and methods of the invention, including antibodies, peptides, peptidomimetics, chemical compositions, etc., are all useful for treating subjects either having or at risk of having an immunodeficiency virus (e.g., HIV) related disorder. AIDS and ARC are preferred examples of such disorders. HIV-associated disorders have been recognized primarily in “at risk” groups, including homosexually active males, intravenous drug users, recipients of blood or blood products, and certain populations from Central Africa and the Caribbean. The syndrome has also been recognized in heterosexual partners of individuals in all “at risk” groups and in infants of affected mothers.

Retroviruses have been linked to a wide range of diseases, including anaemia, neurological disorders, immune suppression, and malignancy. HTLV-I, for example, is associated with tropical spastic paraparesis, a condition similar in some respects to multiple sclerosis. As used herein, “tissue” means any tissue in which pathologic changes, such as with an infection can exist. The term tissue also encompasses the cells of a tissue, for example, a mucosal tissue is composed of mucosal cells. Such tissues and cells include, for example, circulating, isolated and/or cultured cells, gastrointestinal tissues (e.g., the small intestine, the large intestine, the rectum), uro-genital tissue (e.g., vaginal tissue, penile tissue, urethra), nasal-larynx tissue (e.g., nasal tissue, larynx tissue), liver tissue and hepatic cells, and skin (including keratinocytes, fibroblasts) to name a few. Other tissues, including mucosal tissues, are known and easily identifiable by one of skill in the art.

The inventors have discovered that viral infections (e.g., immunodeficiency viral infection such as HIV infections) are an inflammatory condition present in the tissue of a subject (e.g., mucosal tissue). Most reports have emphasized a state of lymphopenia or “anti-inflammation” in the mucosa paralleling that seen progressively in the blood of infected subjects. Because mucosal tissue is populated by an increased number of activated, memory, co-receptor expressing CD4+ T cells in healthy uninfected individuals, the vulnerability to infection is high. In a typical response to infection, mucosal immune cells (most likely CD8+ T-lymphocytes and macrophages) secrete increased levels of pro-inflammatory chemokines and cytokines with the intent of recruiting additional T-lymphocytes to the mucosal site. This heightened response or “inflammatory response”, although instigated with the intent of limiting infection, serves to provide significantly increased numbers of potential new targets for infection, favoring spread of the virus and maintaining the viral burden in these tissue compartments.

For example, in SIV-infected macaques, the gastrointestinal tract is the major site of early CD4+ lymphocyte depletion and viral replication to such an extent that it has been suggested that SIV infection may primarily be a disease of the mucosal immune system (Veazey et al, Science 280:427 (1998); MacDonald and Spencer, Gastrointestinal and Hepatic Immunology, R. H. Heatley, Ed. (Cambridge University Press, 1994). In humans, HIV infection also involves the mucosal immune system and infectious viral particles have been recovered directly from mucosal samples and in situ studies have demonstrated that lamina propria T lymphocytes are among the first cells that are infected (Koteler et al., Am. J Pathol. 139:823 (1991); Heise et al., J Infect. Dis. 169:1116 (1994); Heise et al., Am. J Pathol. 142:1759 (1993); Smit-McBride et al., J Virol. 72:6646 (1998); Clayton et al., Gastroenterology 103:919 (1992); Ellakay et al., Am. J Clin. Pathol., 87:356 (1987); Jarry et al., Histopathology 16:133 (1990); Lacner et al., Am. J Pathol. 153:481 (1998). Moreover, the mucosal lining of the rectosigmoid colon is a primary site for viral introduction during anal-insertive intercourse (Patterson et al., Am. J Pathol. 153:481 (1998)).

The intestine, even in healthy HIV-uninfected patients, maintains a state of low-level physiologic inflammation that is necessary to protect the interior milieu from the bath of potential pathogens that contact its surface. The great majority of lymphocytes and macrophages that compose this infiltrate are therefore stimulated or activated. The primary target of HIV, in which the virus most effectively replicates, is the stimulated CD4+ cell. This type of cell fills the gastrointestinal mucosa. Increased viral replication results in greater spread of HIV throughout the mucosa and higher mucosal HIV viral loads. The predominant aim of anti-HIV therapy is to decrease the ability of HIV to replicate and therefore spread amongst CD4+ cells which are eventually destroyed by the virus.

The gastrointestinal mucosa is one element of this lymphoid tissue and increasing evidence suggests that HIV involves the mucosa at all stages of disease. Not only is the gastrointestinal tract the route of transmission for the majority of patients, but it is the largest lymphoid organ (e.g., a gut-associated lymphoid tissue). As mentioned above, the gastrointestinal mucosa is characterized by a state of low-level physiologic inflammation, and the majority of its lymphocytes are activated. The naturally high concentration of pro-inflammatory cytokines that are present in the mucosa appear to enhance HIV replication in this site, resulting in a high mucosal HIV viral load and successive rounds of infection of new target gastrointestinal CD4+ cells, regardless of the route of infection.

The inventors have found that the majority of gastrointestinal CD4+ T cells express the chemokine receptors that are necessary for HIV entry. The vast majority of lymphocytes of the gastrointestinal mucosa express both CCR5 and CXCR4. In the case of CCR5, it also appears that the mucosal mononuclear cells (MMCs) express higher levels of this receptor than blood derived monocytes on a per cell basis. The inventors have found that the mucosal cells are more susceptible to HIV than are peripheral blood cells in vitro. Accordingly, the gastrointestinal tissue harbors a majority of the virus. During infection, active virus replicates and is shed by infected cells into the surrounding cellular milleu and is eventually transported into the blood stream. Current techniques measure the presence of the virus through assays on a subject's plasma. Thus, therapeutic treatment of a subject may reduce the amount of replication or activiation of the virus resulting in a concommitant reduction in the amount of viral burden in the plasma. However, this reduction is not indicative of the whole body viral burden as a majority of the virus exists in the tissue compartments of the subject.

HIV nucleic acids can be found in the mucosa of the majority of HIV-infected individuals; Kotler et al. detected HIV DNA by PCR using gag-specific primers in 70% of 20 patients he investigated. The inventors have found that even patients with undetectable plasma viral loads have replicating virus in their mucosa. A high SIV viral load is seen in the gastrointestinal mucosa whether the macaque is infected via the gut or via the parenteral route suggesting that the mucosa has a high intrinsic susceptibility to SIV or HIV. After infection, these macaques exhibit a profound early (within 7 to 21 days) loss of gastrointestinal mucosal CD4+ cells. This sign of vigorous HIV activity was not mirrored in other lymphoid sites. In humans, mucosal CD4+ cell depletion has been described in the colon and duodenum during both the early asymptomatic phase of chronic infection and after the onset of clinical AIDS.

Highly, active antiretroviral therapy (UAART) has the potential to drive plasma viral load (PVL) to below detectable levels in some patients as defined by current levels of detection, as well as future definitions of plasma detectability. Yet substantial amounts of HIV remain in these patient's tissues and this viral population eventually undercuts the effectiveness of HAART. As a result, it is widely acknowledged that HAART as it is currently practiced cannot cure AIDS.

The limitations of plasma viral load as discussed above, have two ramifications. First advances in the treatment of HIV disease will require assays that reflect viral parameters in tissue compartments. The lowest limits of detection in PVL tests are attainable by current treatment strategies, although these strategies fail to suppress HIV completely. Second, since many of the processes underlying the disease occur in tissue compartments, an understanding of the disease will require the ability to access viral dynamics in these compartments. Accordingly, the present invention provides methods and compositions useful in assaying the viral loads in these compartments. When used in combination with therapeutic agents such methods and compositions are hopeful in further affecting the control of HIV replication, activation and ultimately reduce the incidence of AIDS.

Since the limits of PVL tests are reached by treatments that do not cure the disease, these tests are unable to determine the effect therapeutics might have on the viral load in various tissue compartments outside of the plasma and blood.

Accordingly, the present invention provides a method for assaying viral load in these tissues in order to better quantitate the viral load (e.g., whole body viral load) as well as the effect of therapeutic treatments in reducing viral load in tissues outside of the blood and plasma as well as viral load in the whole body during therapeutic treatment. Such methods include the potential to determine whole-body viral burden or the total amount of viral load (e.g., HIV viral load) in a subject. By “subject” is meant any mammal, preferably a human. Such determination will utilize measurements from various samples (e.g., tissue or cells) obtained from a subject. For example, tissue, cell or other sources useful in the present invention include for example, circulating, isolated and/or cultured cells, gastrointestinal tissues (e.g., the small intestine, the large intestine, the rectum), uro-genital tissue (e.g., vaginal tissue, penile tissue, urethra), nasallarynx tissue (e.g., nasal tissue, larynx tissue), liver tissue and hepatic cells, and skin (including keratinocytes, fibroblasts) to name a few. The tissue will then be assayed to determine the viral load based upon the presence of viral proteins or nucleic acids (e.g., DNA and RNA).

While highly active anti-retroviral therapy (HAART) may reduce viral load to undetectable levels in the plasma, the vast majority of patients will suffer a rebound increase in plasma viremia when therapy is halted, suggesting that reservoirs of virus exist beyond the bloodstream. One viral reservoir has been discovered in lymphoid tissue where the majority of the body's lymphocytes reside (98%). Since the gastrointestinal mucosa contains the majority of the body's lymphocytes (40-65%), it likely represents the largest reservoir of HIV, and should be considered a primary target for anti-HIV therapy.

The use of HAART by the majority of HIV-infected patients has resulted in prolonged life expectancy of HIV-infected patients. Unfortunately, despite a reduction of plasma load to undetectable levels, multiple studies have shown continued HIV replication in lymphoid organs. Studies have shown that 88% of patients with undetectable plasma viral load have quantifiable HIV nucleic acid in their gastrointestinal mucosa. For example, mucosal biopsies can be used for mucosal mononuclear cell (MMC) isolation, Rnase protection assay (RPA), PCR for HIV RNA and proviral DNA as well as quantitative image analysis (QIA).

Also provided are methods for determining the effect of a therapeutic on whole body-viral load. For example, subjects with plasma detectable or undetectable HIV, having undergone baseline biopsies are treated with a therapy. At various times during the therapy, the subjects will undergo repeat endoscopic biopsies and repeat phlebotomy to obtain PBMCs and biomolecular experiments performed to quantitiate various parameters in the sample, including the presence or absence of particular nucleic acids (e.g., viral DNA or RNA), particular proteins, polypeptides, or antigens (e.g., env protein of HIV) as well as others commonly used to determine the presence or absence of various viral markers. In addition, and as an example, one biopsy at each time point will be utilized for analysis of the patients tissue viral load and plasma obtained by phlebotomy at each time point will be analyzed by the Roche ultrasensitive assay (detection level<40 copies of HIV RNA). RT- and DNA-PCR are performed to analyze changes between baseline and post-therapy tissue and plasma viral load. For statistical evaluation, the solitary index of efficacy will be a decrease in tissue viral load without a concomitant increase in plasma viral load.

Accordingly, the present invention provides a method for assaying viral load in these tissues in order to better quantitate the total body viral load as well as the effect of therpaeutic treatments in reducing viral load in tissues outside the blood and plasma. Such methods include determining whole-body viral burden or the total amount of HIV in a subject. By “subject” is meant any mammal, preferably a human. Such determination will utilize measurements from various tissue sources in a subject. For example, tissue sources useful in the present invention include gastro-intestinal tissues (e.g., the small intestine, the large intestine, the rectum), uro-genital tissue (e.g., vaginal tissue, penile tissue, urethra), nasal-larynx tissue (e.g., nasal tissue, larynx tissue) to name a few. The tissue will then be assayed to determine the viral load based upon the presence of viral proteins or nucleic acids (e.g., DNA and RNA).

Thus, in one embodiment, the present invention provides a method for determining the viral load in a sample obtained from a subject. The sample may be obtained by any number of methods including, for example, endoscopically or percutaneously, as well as other methods known in the art. In an endoscopic embodiment a flexible tube (e.g., an endoscope) is inserted into an orifice, such as the rectum, which allows a technician, physician or nurse to examine the rectum and use a forceps to obtain a biopsy sample. The biopsy sample is typically taken at a distance of about 30 cm and is about 8 mm in diameter. The tissue biopsy is then processed, using standard techniques known to those of skill in the art, to extract nucleic acids (e.g., RNA and DNA). The RNA is converted to DNA by reverse transcriptase and amplified by PCR in order to quantify the presence of a virus (e.g., HIV by detecting HIV RNA) in the sample. In another embodiment, the quantity of HIV nucleic acids in the tissue sample are compared to a standard sample or directly amplifed by PCR to quantify DNA. Alternatively, the quantity of viral nucleic acids in the tissue can be compared to the amount of viral nucleic acids in a corresponding plasma sample obtained from the same subject. This later embodiment allows for the determination of the effect of antiviral treatment on tissue viral burden, blood and rectal biopsy specimens in order to determine the effect of therapy on the various viral loads in each sample. An additional embodiment is the quantitiative monitoring using internal controls of nucleic acid decay in each sample from the time of acquisition through the assay in order to ensure that detected levels accurately correlate with the in vivo status of the sample

In another embodiment the invention provides a kit for obtaining samples from a subject comprising a collection device, such as a forceps or scapel, a cell lysis buffer suitable for preserving nucleic acids in the biological sample, and a spiking agent, such as a nucleic acid (e.g., RNA or DNA). By “spiking agent” is meant a DNA or RNA sample of known amount that degrades at a known rate, such that one skilled in the art can determine the rate of change in any particular sample or in any particular molecule within a sample. Such a kit may also include a carrier means being compartmentalized to receive in close confinement one or more containers such as vials, tubes, and the like, each of the containers comprising one of the separate elements to be used in the method. If present, a second container may comprise a lysis buffer. The kit may also have containers containing nucleotides for amplification of or hybridization to a specific nucleic acid sequence which may or may not be labeled, or a container comprising a reporter, such as a biotin-binding protein, such as avidin or streptavidin, bound to a reporter molecule, such as an enzymatic, florescent, or radionuclide label.

### EXAMPLES

Tissue-based immune cells can be easily and safely obtained and isolated from the mucosal lining of the gut, a renewable tissue source. As studies of HIV-1 pathogenesis increasingly focus on tissue compartments for both persistence and transmission studies, techniques to easily and safely access lymphoid tissue are essential. Lymph node and tonsillar resection/aspirations have been the most commonly reported methods. Studies using these approaches have already provided illuminating, concept-changing findings including evidence that HIV-1 activity persists in lymphoid tissue when plasma levels are stable or undetectable. These tissue sources reveal the biologic events that occur in secondary, organized lymphoid structures during HIV-1 infection, but require invasive surgical support for tissue acquisition. In contrast, the gut mucosal lymphoid tissue is abundant, easily accessible, quickly healing, self-replenishing, and directly visible. Endoscopic biopsies are safe, quick, painless, and provide access to the lymphoid compartment with 100% of samples containing lymphocytes. The biopsies maintain architectural orientation and can be examined histologically, or can be dissociated for flow cytometric and tissue culture evaluation as described here.

Several examples have been presented in FIG. form to demonstrate representative clinical utilities of this invention. The invention has been used to quantify tissue viral burden (RNA and DNA) in (i) a study of 20 subjects with persistently suppressed plasma viral burden (<400 copies/ml plasma) every three months for 1 year; (ii) a study of 5 subjects with detectable plasma viral HIV RNA undergoing a trial of gene therapy in which tissue sampling was utilized to quantitate changes in tissue viral burden; (iii) a study of 40 subjects with undetectable plasma viral burden (<40 copies per ml plasma) undergoing gene therapy for their HIV infection in which gene therapy was used to detect changes in tissue HIV RNA and DNA as primary endpoints; (iv) a group of 8 subjects with detectable plasma viral HIV RNA to determine patterns of tissue genotypic and phenotypic resistance based on tissue-extracted nucleic acids; (v) a group of 10 subjects with undetectable (<40 copies per ml plasma) to determine tissue levels of HIV RNA and HIV DNA as well as tissue patterns of genotypic and phenotypic resistance.

For all studies, informed consent (approved by the UCLA IRB) was obtained prior to enrollment. Routine flexible sigmoidoscopy was performed and a standardized site of 30 cm in the rectosigmoid colon was routinely used for all sampling to avoid potentially confounding inflammation resulting from traumatic or infectious proctitis. Biopsies were collected using 3.3 OD forceps (8 mm open span). For nucleic extraction studies, samples are removed from the forceps in to pre-labeled cryovials and immediately suspended in liquid nitrogen (<15 seconds from acquisition to freezing) for transport to the laboratory. At that point, samples are transferred into -80° C. freezer and data entered into a 4dimensional relational database.

For biopsies intended for isolation assays to obtain mononuclear suspensions of mucosal cells, acquired biopsies are maintained at room temperature. Informed consent was obtained prior to undergoing elective endoscopy for a history of blood in stool or routine polyp screening. No subjects had diarrhea symptoms or history of intestinal inflammatory or infectious disorders. Hematoxylin and eosin stained biopsies taken in the same area as study biopsies revealed no pathology and were all normal appearing when reviewed in a blinded fashion by a gastrointestinal surgical pathologist. The study was approved by the UCLA Human Subjects Protection Committee. A site of 30 cm in the rectosigmoid colon was used routinely for all sampling to avoid potentially confounding inflammation resulting from traumatic or infectious proctitis. Mucosal mononuclear cells (MMC) were isolated from four endoscopic biopsies from each donor. Biopsies were collected using 3.3 OD forceps into 15m1 of tissue culture medium (RPMI 1640, Irvine Scientific). The biopsies were maintained at room temperature on a rotating platform until isolation (roughly 20-60 minutes) then removed to a 10×35 mm petri-dish containing phosphate buffered saline (PBS) with 1mM EDTA and 50mM 2-mercaptoethanol and the samples teased apart using 18 G needles. The disrupted tissue was incubated at 37° C. for 20 minutes in a shaking water bath. Following centrifugation, the tissue samples were digested with a mixture of collagenase and dispase (Boehringer Mannheim # 269638; 0.1 mg/mL in RPMI) for 1 hour at 37° C. Further disruption was achieved by sample passage through syringes with a series of decreasing needle gauges. Debris was removed using a 70 micron cell strainer (Falcon # 2350). Resulting cells were resuspended in RPMI containing 10% fetal calf serum. Mononuclear cells, which included primarily epithelial cells and leukocytes, were counted visually using a hemocytometer and the proportion of mononuclear cells that were leukocytes was estimated. About 20% of the mononuclear cells were leukocytes from a yield of mean 1.3×106±1.1×106 S.D. (n=6) per four biopsies. Viability, determined by the exclusion of trypan blue, was >90%. Blood from the donors was collected in EDTA and was stained using the whole blood staining method.

Among the multiple co-receptors that HIV-1 is able to utilize, CCR5 and CXCR4 play a major role, with CCR5-tropic viruses predominating during initial infection, CXCR4-tropic viruses becoming more prevalent with advanced disease and heterozygosity of CCR5 contributing to longer survival. Differential expression of CCR5 on mucosal CD4+ T lymphocytes could contribute to preferential transmission of M-tropic viruses. In order to compare co-receptor expression on mucosal versus circulating lymphocytes, mucosal mononuclear cells (MMC) were isolated from rectosigmoid endoscopic biopsies and obtained unstimulated phlebotomy samples from HIV- 1 seronegative healthy individuals. We quantified co-receptor expression on CD4+ cells by flow cytometry.

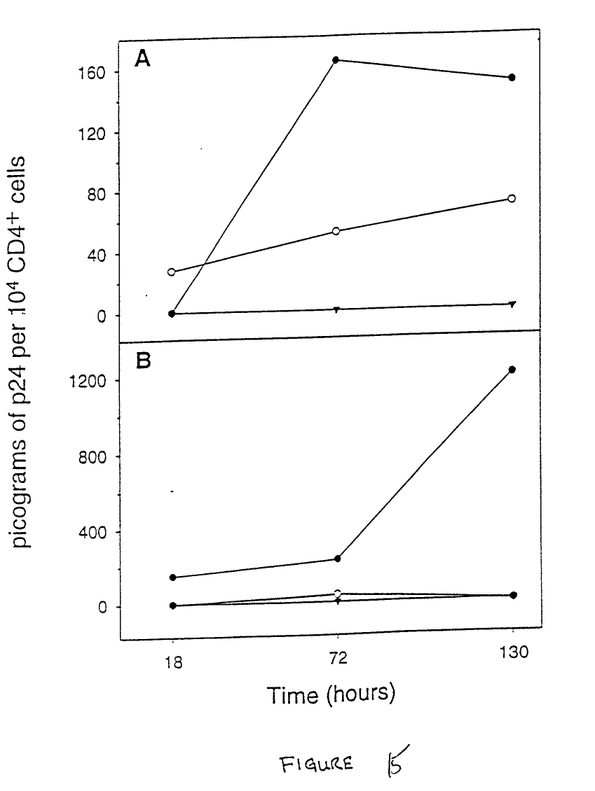

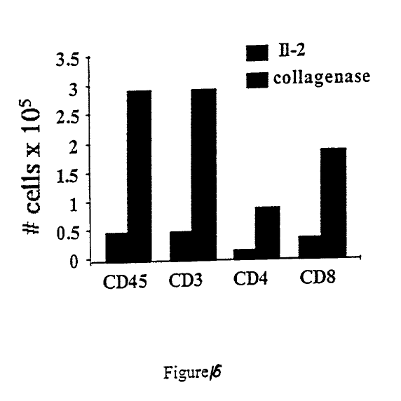

In agreement with published studies, a median of 23% (interquartile [i.q.] range 18-30%) of all CD4+ lymphocytes in blood expressed CCR5. As shown in FIG. 15, a median of 71% (i.q. range 50-87%) of the CD4+ lymphocytes in the gut expressed CCR5, a 2.8-fold greater percentage than in the blood (P=0.03). Mucosal CD4+ lymphocytes also expressed significantly more CCR5 receptors per cell than did their CCR5-expressing CD4+ lymphocyte blood counterparts, further extending the compartmental difference. As shown in FIG. 16, the median CCR5 receptor number per CD4+ mucosal lymphocyte was 6,946 molecules (i.q. range 6,306-10,416) compared to approximately 3,841 (i.q. range 3,259-4,441) CCR5 receptors per CD4+ blood lymphocyte, a 2.2-fold increase (P=.03). Taken together, this translates into a 6.2-fold increase in total expressed CCR5 receptors potentially available for viral access on CD4+ lymphocytes in the gut compared to the blood. These findings suggest that mucosal CD4+ lymphocytes may be much more vulnerable to infection by M-tropic HIV-1 than their blood counterparts.

Nearly all (97%) of the CCR5 expression on CD4+ lymphocytes in both the blood and gut was on cells of the memory CD45RO+ phenotype. In agreement with published studies, we found an increased proportion of CD45RO+ memory cells among gut CD4+ lymphocytes (median 95%; i.q. range 90-97%) compared to blood (median 46%; i.q. range 38-53%). Our findings are based on isolated viable cells using a method found to preserves cell surface expression of CCR5 and CXCR4.

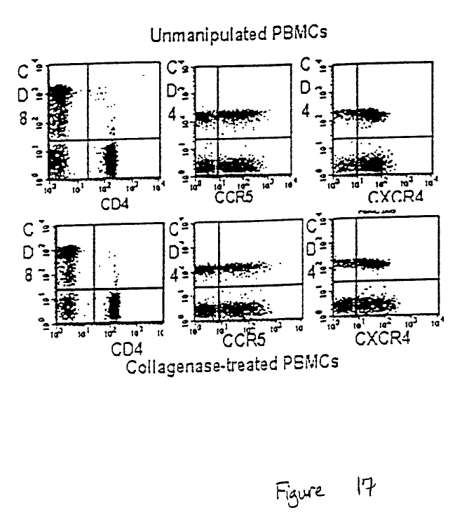

In order to evaluate the susceptibility of mucosal mononuclear cells to HIV-1 infection, we subjected isolated MMC from endoscopic biopsies and isolated PBMCs from healthy HIV-seronegative volunteers to in vitro HIV infection. Infection of mucosal cells with laboratory strains of HIV (M-tropic HIVSX or T-tropic HIVNL4-3) was performed in the presence of 20 IU of interleukin-2 (IL-2), as data had shown that IL-2 was required to maintain viability of mucosal cell populations. As a control, since IL-2 is known to upregulate CCR5 and could enhance viral replication, PBMCs were also infected from the same patient both with and without IL-2. Infection was quantified at 18 hours, 72 hours and 130 hours by p24 production in the supernatant and expressed in terms of pg of p24 produced per 104 CD4+ lymphocytes. Mucosal mononuclear cells were able to support vigorous viral replication in culture compared to PBMC with or without IL-2 as shown in FIG. 17 in a representative experiment (one of two). PBMC infected in the absence of IL-2 could not support HIV replication by either HIVSX or HWNM-4-3. When compared with similarly cultured PBMCs in the presence of IL-2, mucosal cells were markedly more susceptible than PBMC to M-tropic and T-tropic HIV. For example, at 72 hours, supernatant p24 levels of the M-tropic HIVSX in the MMC culture was 164 pg/ml per 104 CD4+ lymphocytes compared with 51 pg/ml in the PBMC culture. For T-tropic HIVNL4-3, viral growth accelerated over time and at 130 hours, supernatant p24 levels in the MMC cultures was 1194 pg/ml per 104 CD4+ lymphocytes compared to undetectable levels in cultures of PBMC. These data indicate that the enhanced vulnerability to infection suggested by the mucosal CD4+ lymphocyte co-receptor phenotype renders them functionally infectable in vitro by both M and T-tropic strains of HIV-1.

These studies is that CCR5 expression is markedly increased on human mucosal CD4+ lymphocytes, both as a percentage of total CD4+ lymphocytes and on a per cell basis compared to peripheral blood cells. These mucosal T cells support higher levels of viral replication than CD4+ lymphocytes from blood (FIG. 17). As CCR5 is the co-receptor most associated with HIV-1 in early infection, and the gastrointestinal tract is one of the most common sites of transmission as well as being the body's major lymphoid organ, the amount of detected CCR5 expression carries important implications for transmission, primary infection, ongoing local spread and treatment. Projections of T cell infectability by M-tropic HIV-1, based on CCR5 expression in blood, would lead to a dramatically different mathematical modeling of disease progression. When CD4 was not a limiting factor, a minimum of 700-2000 CCR5 receptors per cell was adequate for maximal susceptibility to infection. By our calculations, the number of CCR5 receptors on blood T cells (median of approximately 3000 per cell) would be within this range of in vitro infectability. The mucosal levels (median of approximately 7000 receptors per cell) far exceed this minimal range. Factors including b-chemokine production levels and presence of cellular activation factors in the MMC could also impact the capacity of these cells to support replication. Cytokines including TNF-a and IL-2 are found at high levels in gut mucosa and may also contribute to enhanced viral replication at this site.

Our results show that tissue biopsies of gut mucosa can be used to obtain quantitative information on immunologic and virologic determinants that may influence HIV-1 transmission and pathogenesis. The potential vulnerability to primary and persistent HIV-1 infection of the gastrointestinal mucosa, a sexually-exposed and easily traumatized lining, is dramatic. This vulnerability includes the predominance of activated memory CD4+ lymphocytes at this mucosal site. Our results also show that both CCR5 and CXCR4 are highly expressed on mucosal CD4+ lymphocytes from the gastrointestinal tract in healthy, HIV-1 -seronegative individuals, and these mucosal cells are highly susceptible to infection in vitro, much more so than cells from the blood.

To ensure that the collagenase/dispase isolation process used on the gut biopsies did not degrade nor strip surface antigens, peripheral blood mononuclear cells (PBMC) were isolated by Ficoll-Hypaque separation and then either stained and analyzed directly by flow cytometry for percent CD45, CD4, CD80, CCR5 and CXCR4 expression or processed through the mucosal isolation procedure (collagenase/dispase treatment) and then stained and analyzed for the antigens. PBMC routinely isolated and those exposed to mucosal isolation enzymes showed no discernible differences in quadrant percentages for all antibodies studied. Thus, the observation of increased percentages and expression of CCR5 on cells from the gut compared to those from the blood does not result from the isolation process since CCR5 expression was not increased by treatment with collagenase/dispase.

MMCs from each healthy, HIV-seronegative volunteer were isolated from four endoscopic mucosal biopsies after mechanical disruption followed by 3-day culture in Iscove's DMEM medium supplemented with 10% human serum, containing 10 mg/ml gentamycin, penicillin, streptomycin and glutamine. Interleukin-2 (IL-2, Amgen) was added at 20 IU per mL. A total of 105 mucosal mononuclear cells and PBMCs were plated in a 96-well plate in 100 microliters of medium after a 3-hour infection with 50 mg of HIVSX or HWNL4-3. Prior to plating, the cells were washed twice to remove free virus and adherent p24.Thirty microliters of supernatant was sampled at each time point 18 hours, 3 days (72 hours), and 5 days (130 hours) for p24 measurement by ELISA (Coulter). CD4+ percentages were determined by flow cytometry and were used to determine the number of CD4+ lymphocytes in the cultures. Co-receptor expression on the gut cells was not tested on these donors because cells yields were not sufficient.

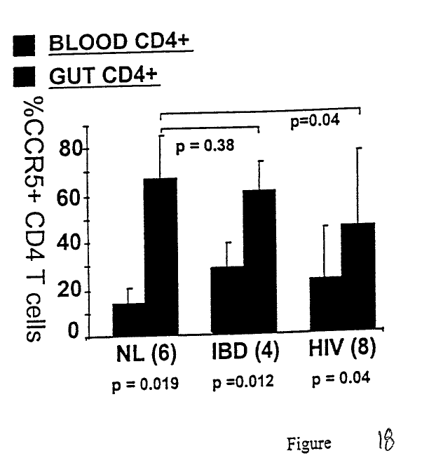

In an attempt to increase the yield of mucosal mononuclear cells for functional, infectivity and flow studies, an alternative isolation method was undertaken. Freshly collected endoscopic biopsies are minced directly into 10ml of Iscoves Medium supplemented with 20 units/ml of IL-2 in a 100×300 mm petri-dish and cultured for 3 days in 5%CO2 at 37 degrees. Cells are harvested through a 70 μm cell strainer and the total mononuclear cell yield enumerated visually by hemocytometer. The yield of CD45+, CD3+, CD4+ and CD8+ cells was determined using TruCount beads and was compared to the yield from biopsies collected from the same individual isolated using the conventional collagenase/dispase protocol. There is a 6-fold increase in the yield of mucosal lymphocytes. (FIG. 18).

### Quantitation of HIV-1 in tissue

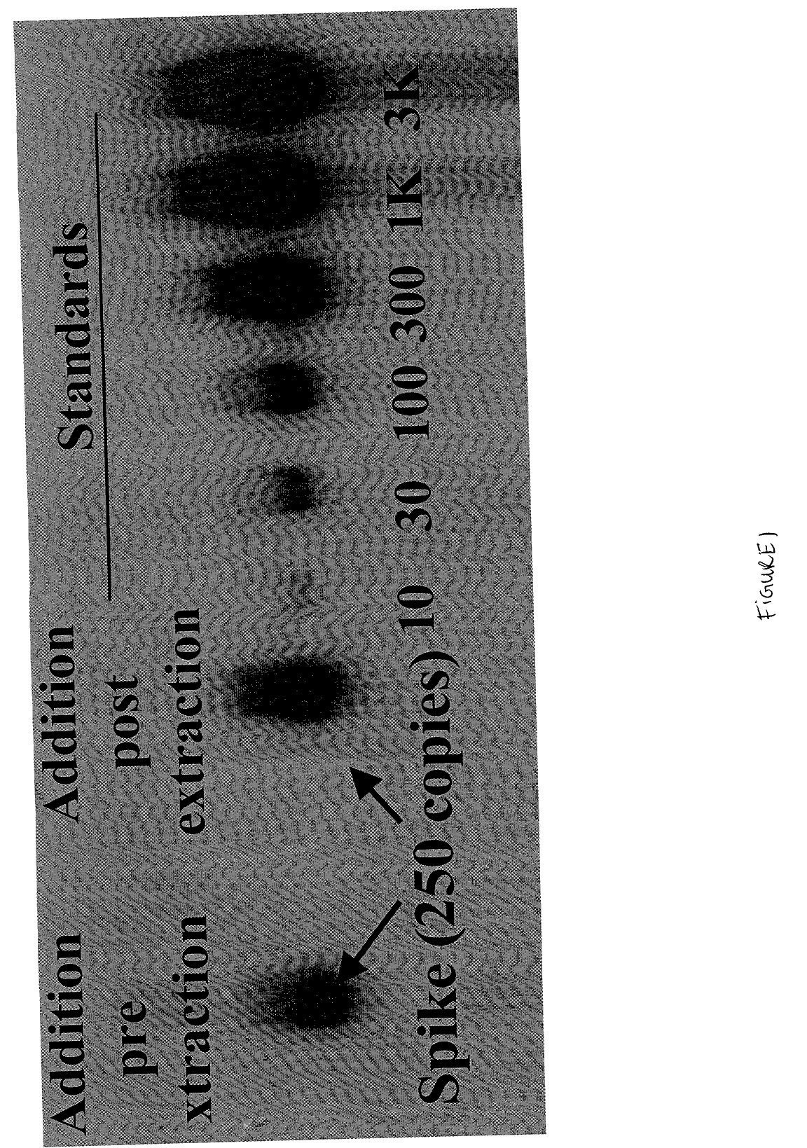

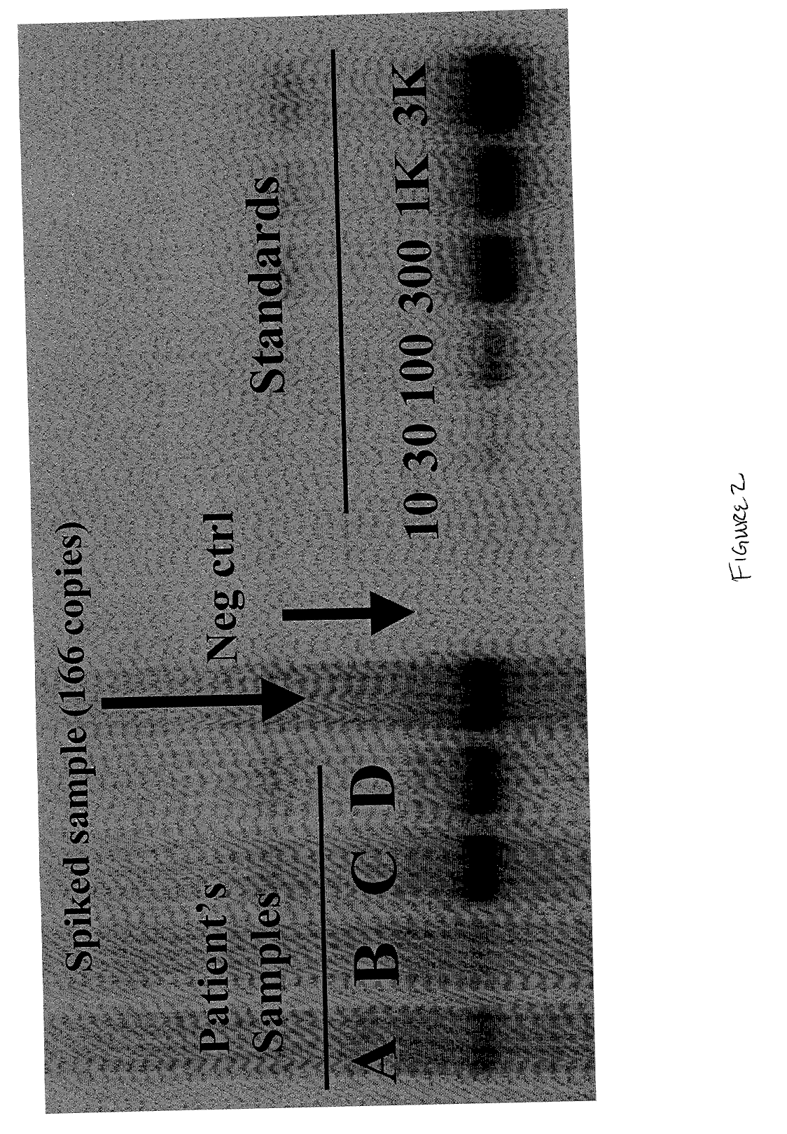

Extraction of RNA from rectosigmoid biopsies results in >95% recovery of tissue RNA. We have developed a quantitative RT PCR assay for tissue RNA and quantitative PCR for tissue DNA, adapted from that previously described by the Chen laboratory. Our initial results show that HIV-1 RNA can be quantitated by RT PCR to levels as low as 10 copies (FIG. 1).

Samples are immediately homogenized (using Powergen 125 tissue homogenizer)or pulverized using a mortar and pestle from the frozen state, Trizol-extracted with separation of RNA and DNA containing phases. RNA is further extracted using an Rneasy column. Quality control studies have confirmed minimal RNA degradation (agarose gel electrophoresis) and no DNA contamination (PCR of RNA template). The number of HIV RNA copies is quantitated using an adaptation of the rTTH RNA PCR kit (Perkin-Elmer) with HIV LTR specific primers 667/AA55 designed to capture unspliced/multiply spliced HIV RNA. A linear standard curve is generated using a 127 bp sequence recognized by the 667/AA55 primer pairs. DNA is isolated by ethanol precipitation with at least 2 washes in 0.1M sodium citrate/10% ethanol buffer. For HIV DNA, the same primer pairs are used for PCR amplification (667/AA55). Linear standard curves have been generated using β-globin primers.

In an effort to standardize our approach and have resultant yields most closely reflect in vivo amounts of HV RNA, known quantities of HIV LTR RNA were added to sero-negative biopsies both pre and post nucleic acid isolation. A linear standard curve was generated using purified HIV LTR RNA diluted in 0.5ug/ul of Hela-cell total RNA and RT-PCR was performed as previously described. The difference between the pre and post LTR- supplemented samples was quantified and found to be minimal (>95% recovery).

As DNA and RNA are extracted from the sample (and same total sampel voluem) and equally divided with internal tracer controls (e.g., luciferase DNA and cylcophilin RNA), the amount of cells in the DNA sampel can be determined using the housekeeping gene β-globin, corrected for loss (via luciferase tracking (“spiking”)); the number of cells supporting the RNA assay can then be indirectly inferred and corrected for RNA loss.

For quantitative assessment of recovery, known quantities of luciferase DNA, a bacterial sequence with no known human homology, to quantitate tissue DNA recovery (usually >75%); sero-negative samples receive a known quantity of LTR HIV sequence to quantitate RNA recovery (>95%).

### HIV-RNA is reproducibly detected in rectosigmoid biopsies from subjects with undetectable plasma viral load.

Efforts were made to demonstrate the replicability of results from one biopsy by comparison with others concurrently obtained at the same circumferential level (30 cm) from the same patient. Single biopsies (10 mg each) from subjects with undetectable plasma viral loads were frozen, RNA extracted and amplified using LTR-specific primers 667/AA55, as described above. Each biopsy yielded an average of 25 μg RNA of which usually 1/100 was used for quantitation. Results in FIG. 2 demonstrate the reproducibility of quantitated RNA viral burden using rectosigmoid biopsies in a sensitive assay. The data demonstrates the tissue HIV RNA viral load from 2 biopsies obtained during the baseline sigmoidoscopies for subjects with undetectable plasma viral loads. These individuals reported undetectable plasma viral load for >1 year. On average, there is <0.2 log10 difference between samples within the same subject. These data show the reproducibility and minimal sample to sample variation in using biopsies to quantitate tissue viral load. Equally important is the demonstration of detectable levels of tissue HIV RNA (usually 102 to 103 per ,g RNA) in subjects with undetectable plasma HIV RNA.

### HIV-DNA is reproducibly detected in rectosigmoid biopsies from subjects with undetectable plasma viral load.

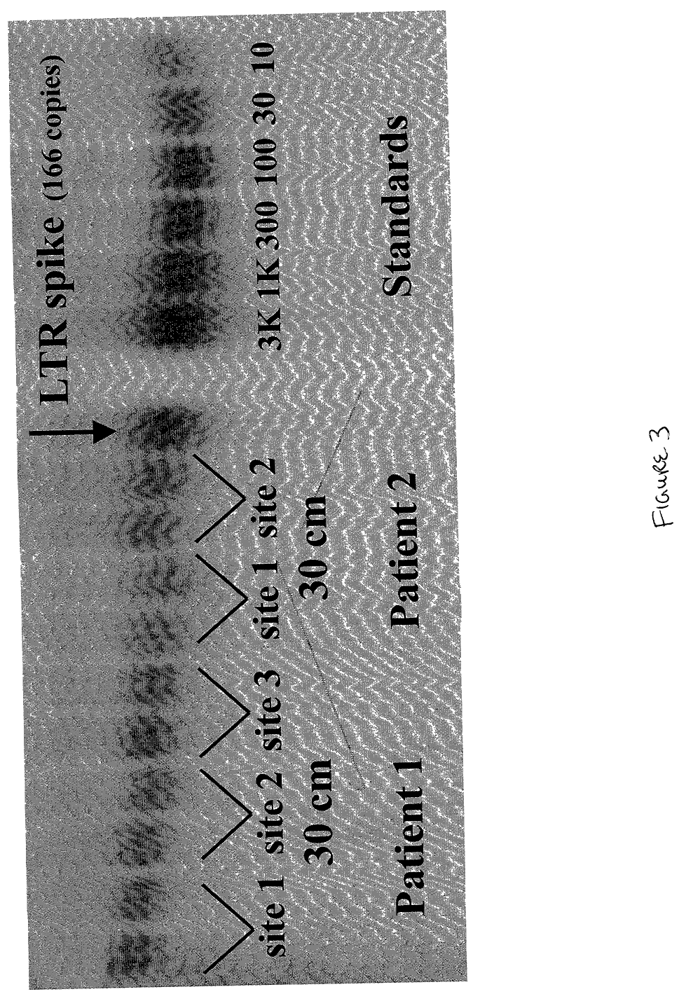

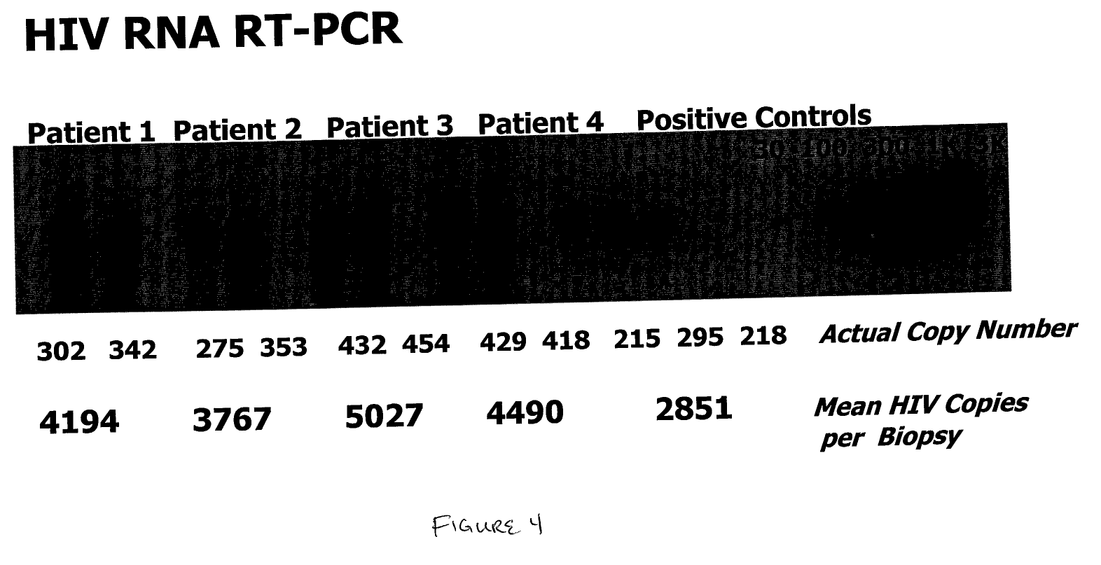

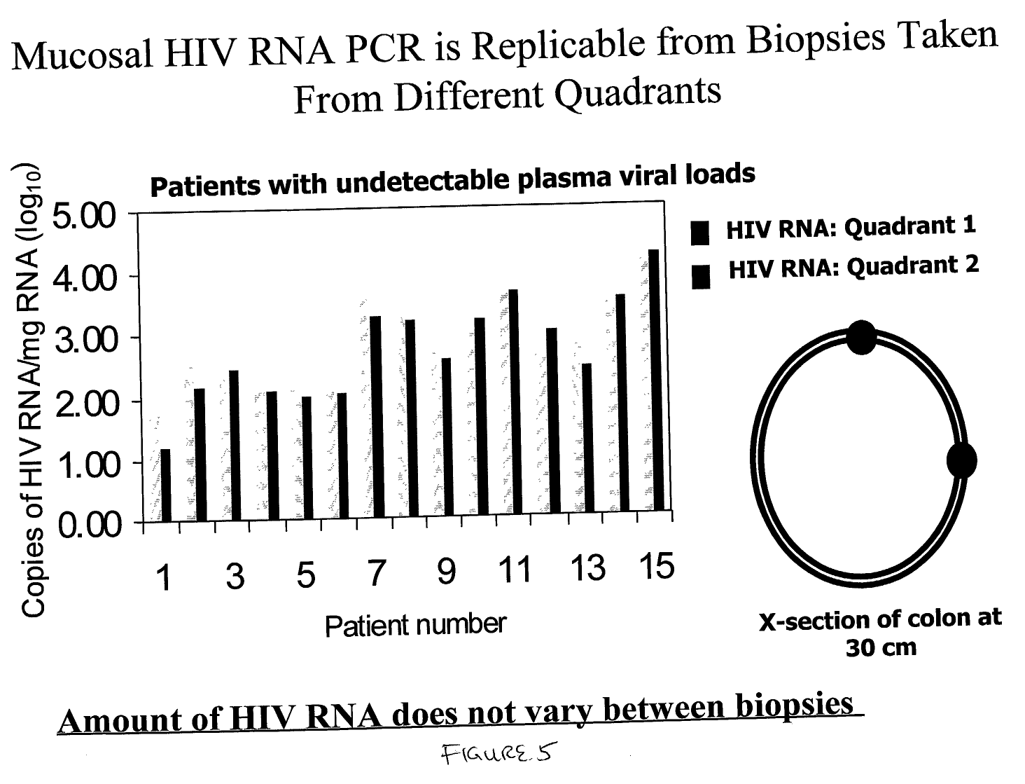

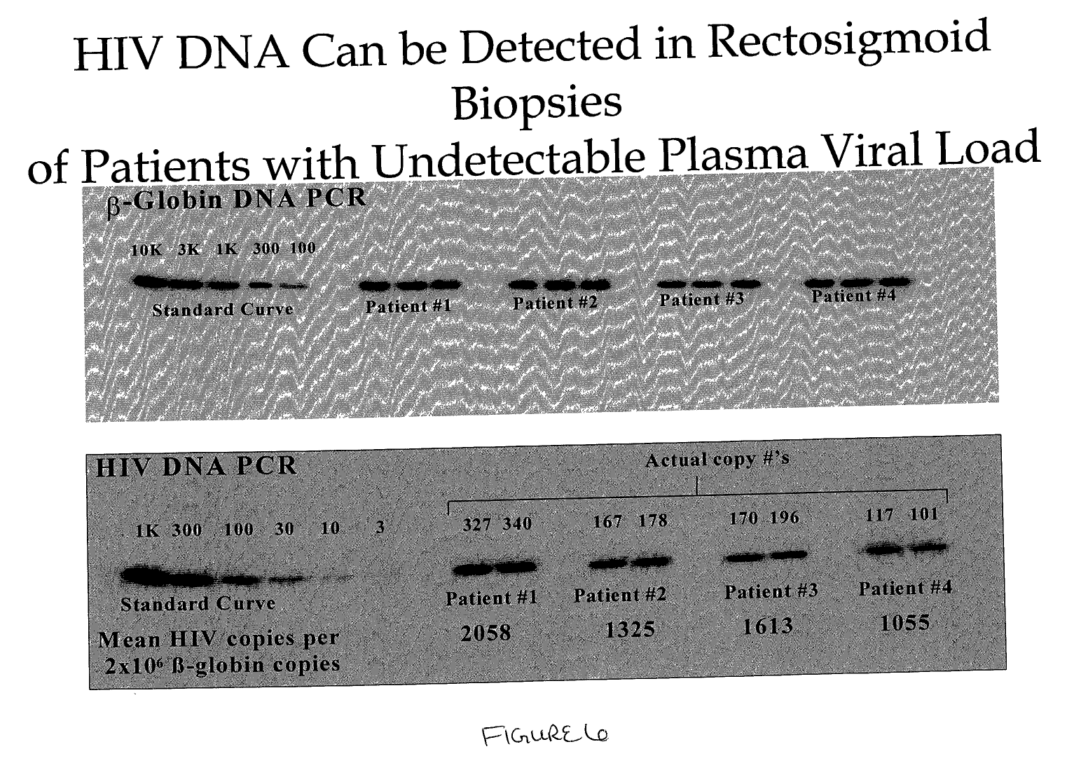

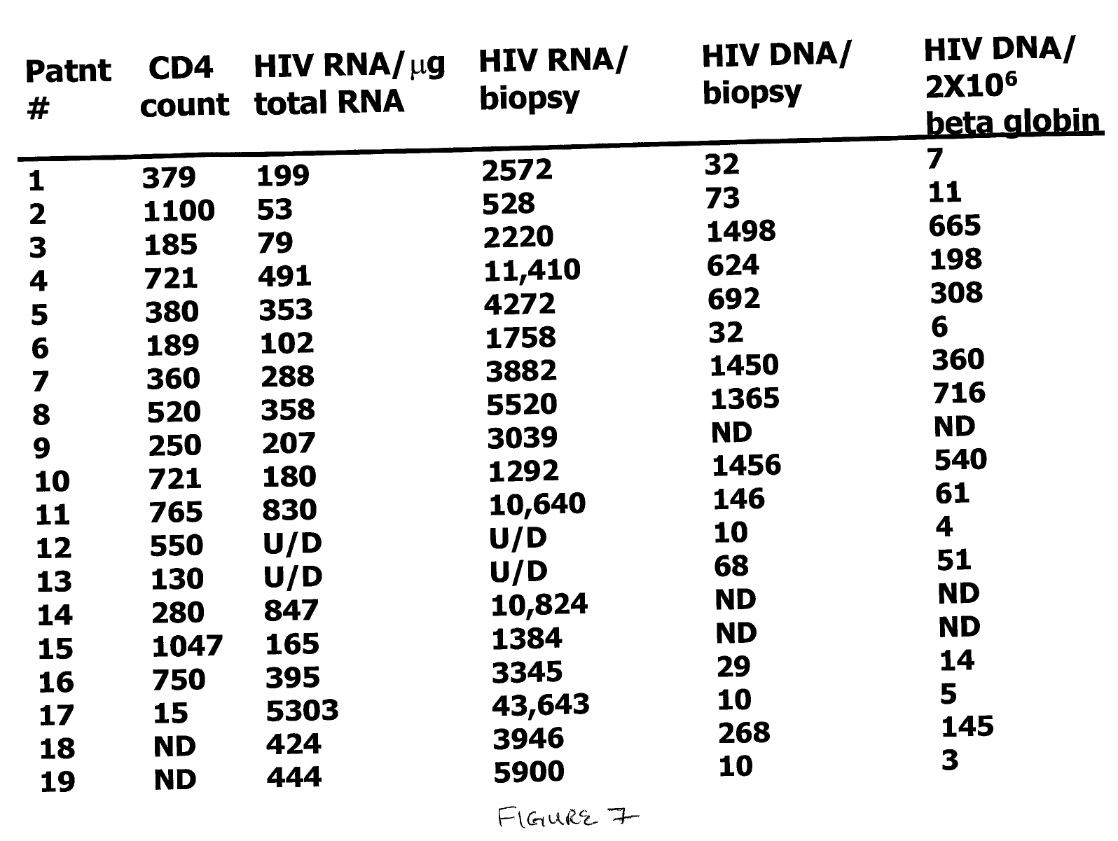

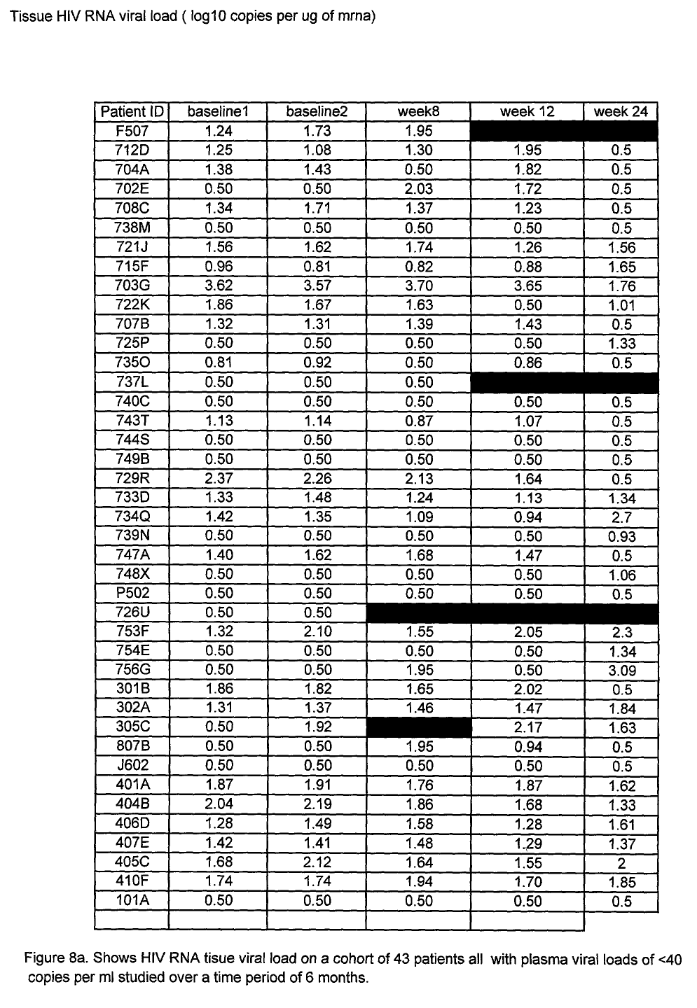

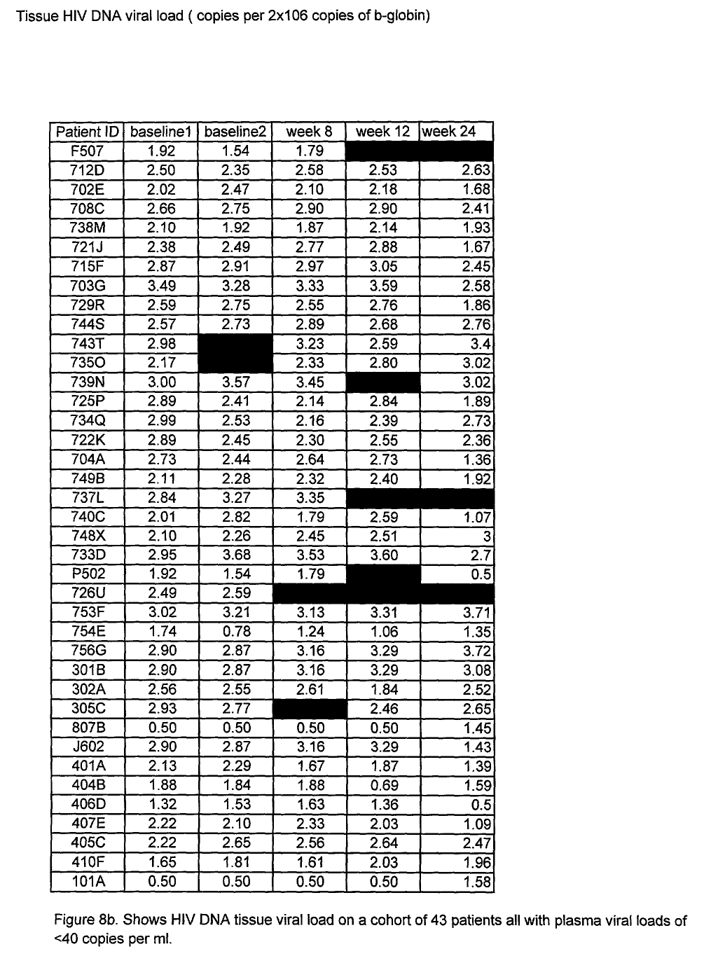

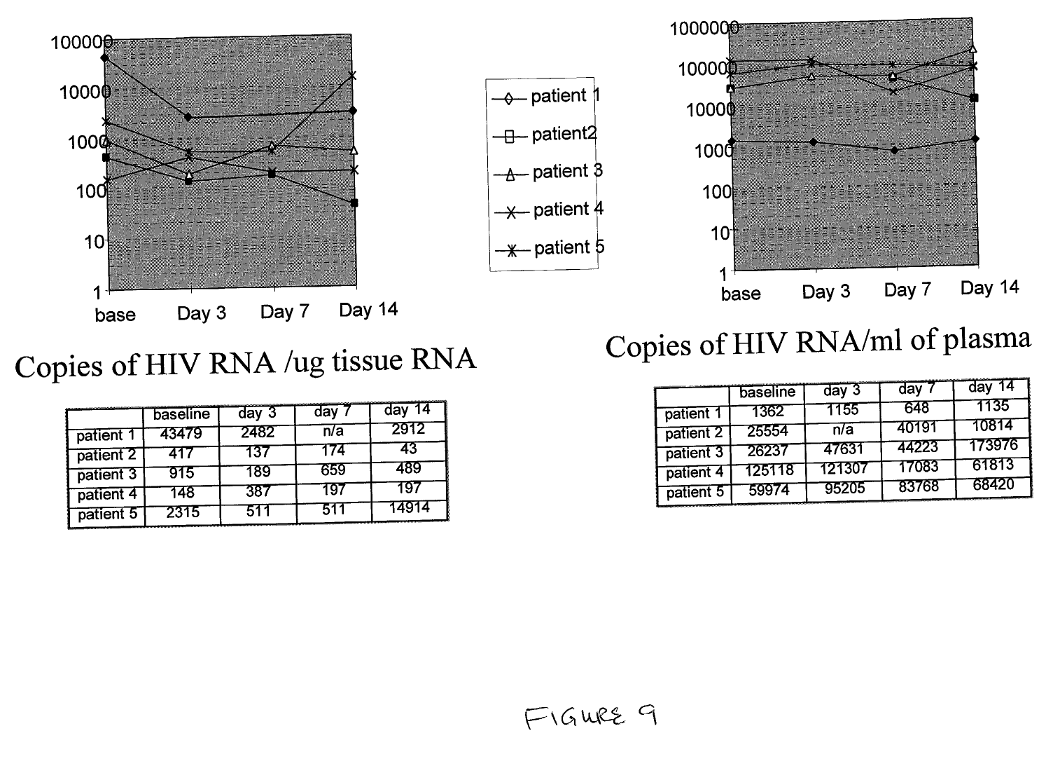

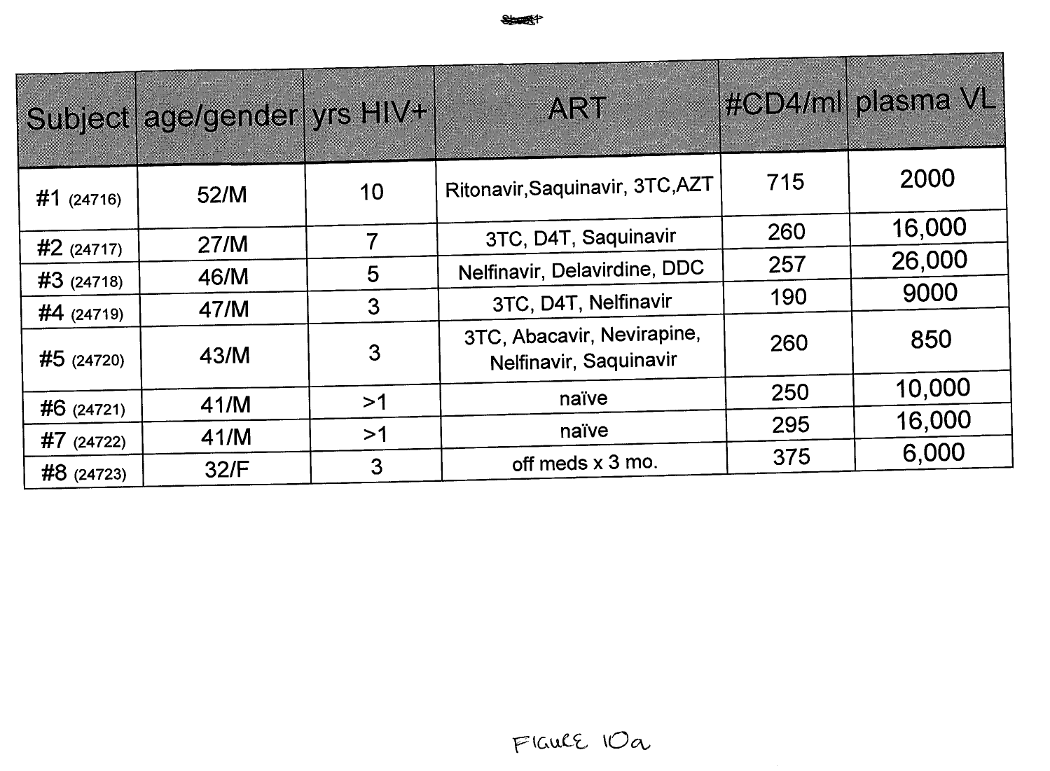

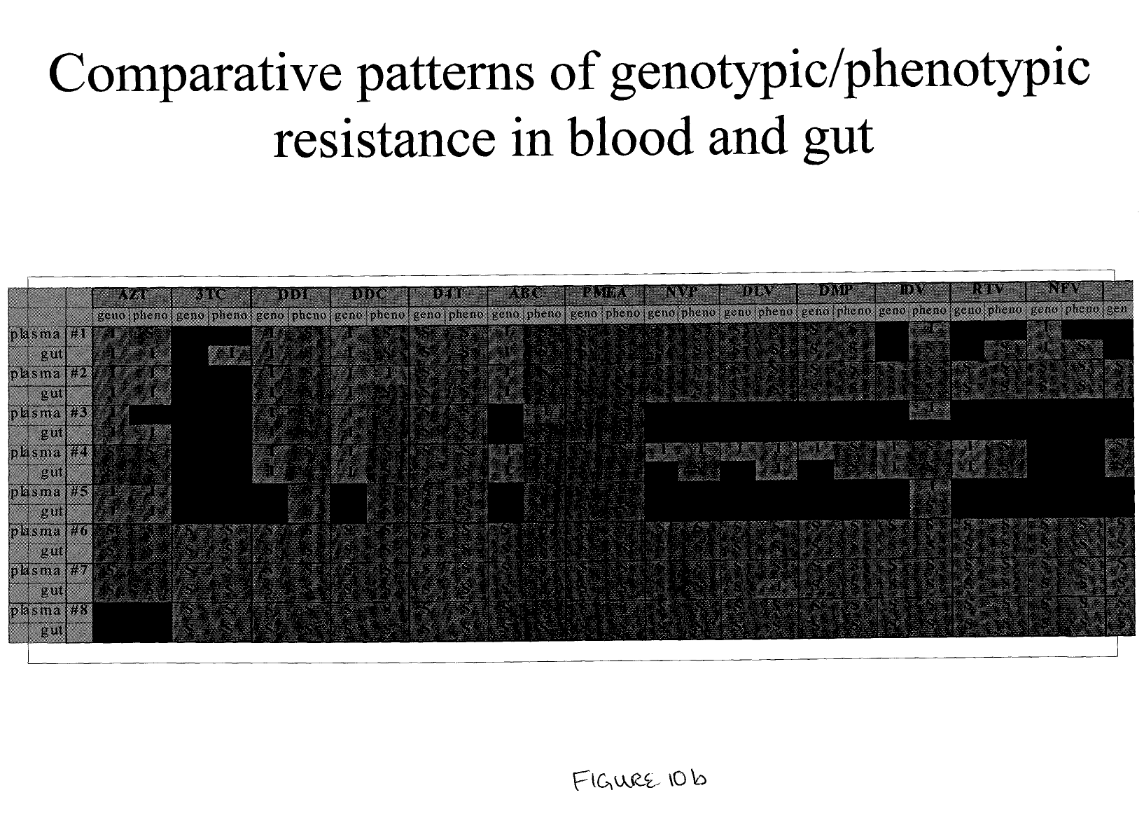

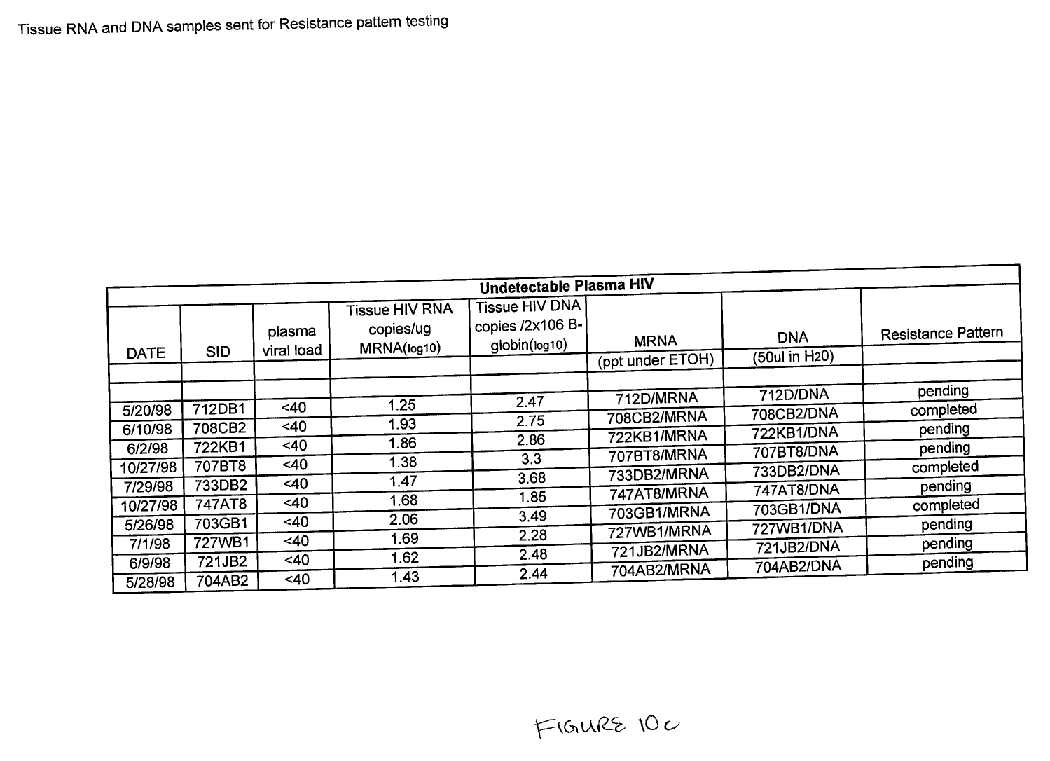

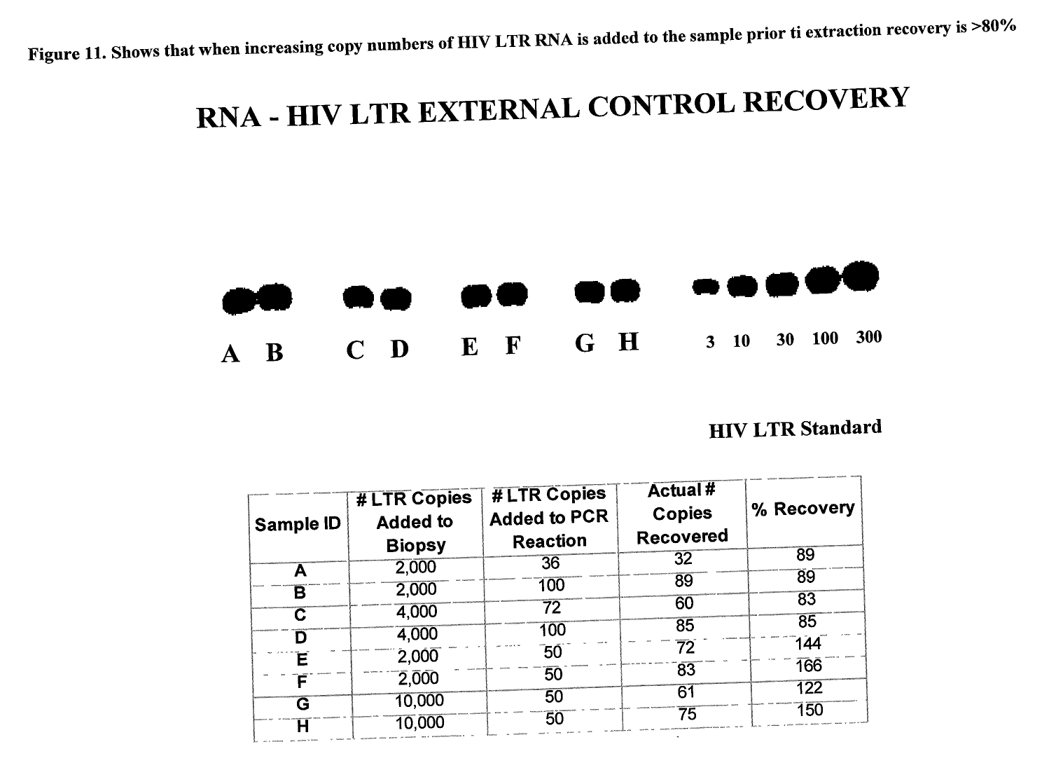

In a separate group of subjects with undetectable plasma viral load, HIV DNA was amplified using quantitative PCR with specific 667/AA55 primers for the LTR region and b-globin specific primers used for internal linear standard. Triplicates were assayed for the b-globin specific primers; duplicates of the HIV proviral DNA quantitation are shown in FIG. 3. Although lower limit of detection is 3 copies, 10 copies were used as our lower cut-off point. The figure shows both the actual copy number quantitated and the calculated number of copies based on a b-globin-dependent cell count. These results show our technique can detect copies of proviral DNA as low as 10 in subjects with undetectable plasma viral load.

### Nucleic Acid Isolation.

Total RNA and DNA is simultaneously isolated from a single endoscopic biopsy by directly homogenizing the tissue using a Powergen 125 homogenizer(Fisher Scientific). Tissue samples are placed into 140 μl of urea lysis buffer and 70 μl of DEPC water in a 50ml conical centrifuge tube (Falcon 2070). Following complete homogenization of the tissue 500ul of saturated phenol (Fisher # 1750-400) is added. An alternative approach to extract RNA and DNA from the same sample is achieved by pulverizing frozen tissue using a mortar and pestle (Fisher Scientific). Equal amounts are then used for DNA and RNA quantitation having been derived from the same piece of heterogeneously mixed tissue.

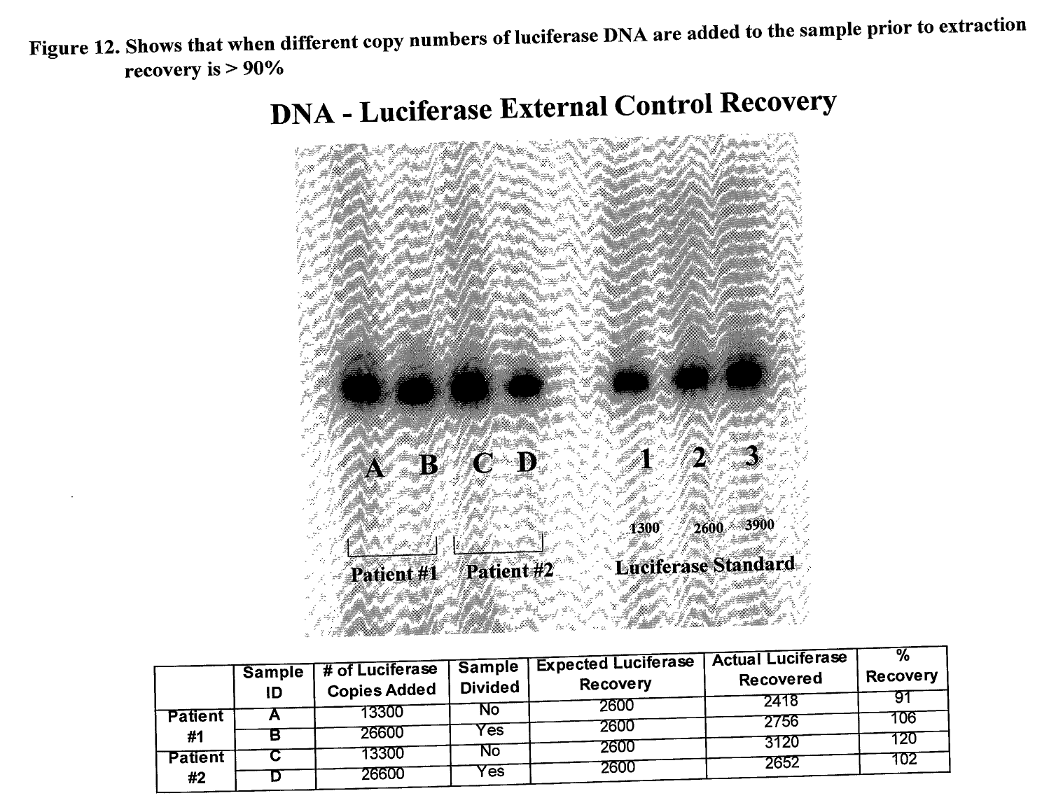

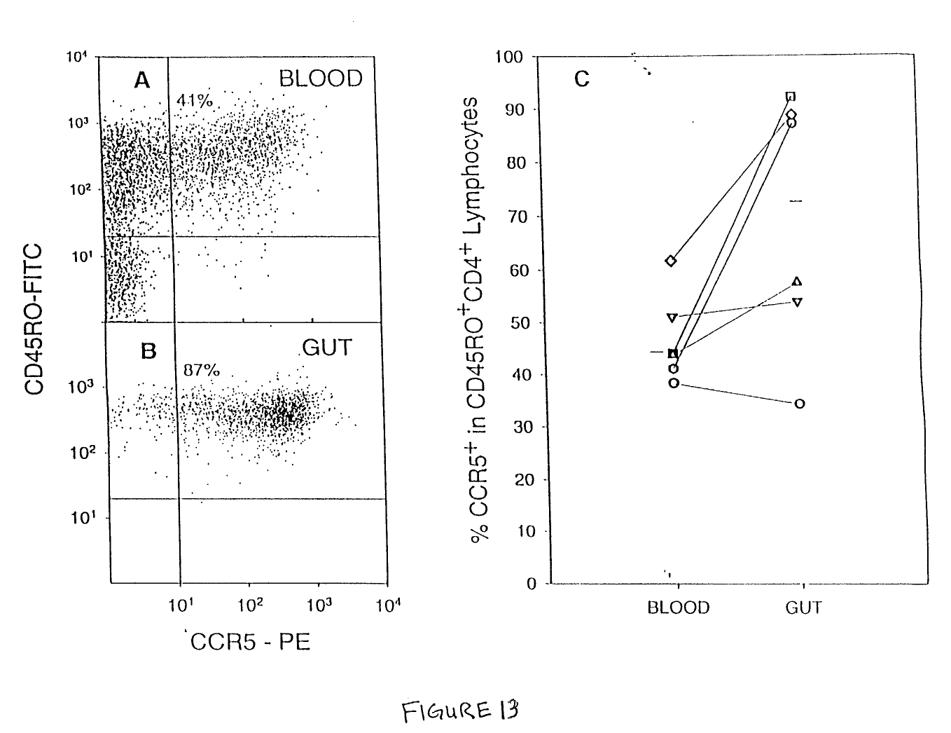

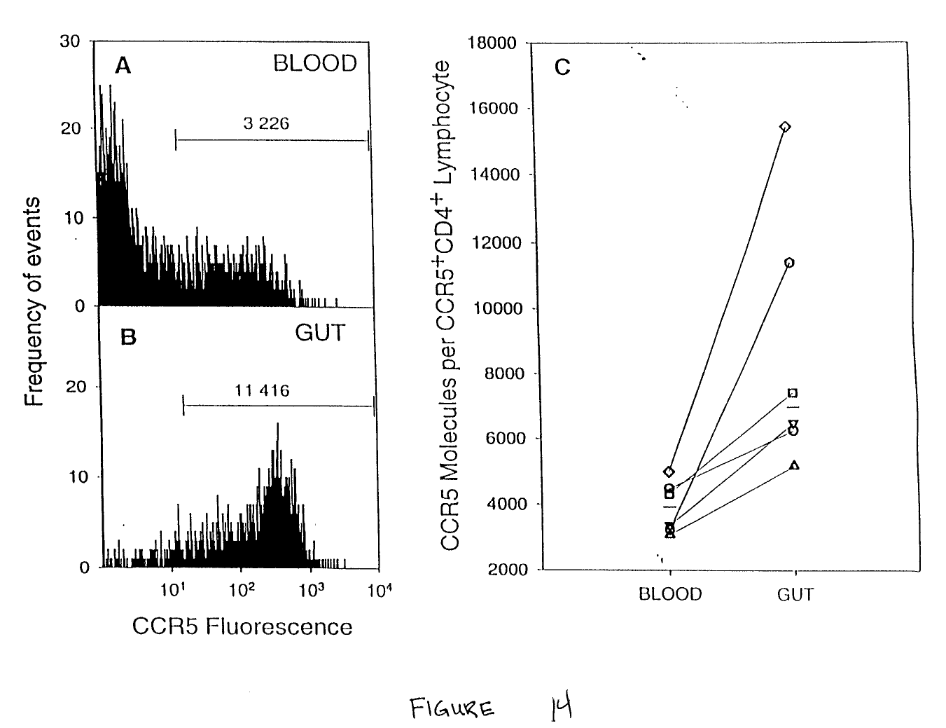

For total RNA isolation 355 μl of the homogenate is added to 1ml of Trizol reagent (Gibco BRL #15596-56) in an Rnase and Dnase free 2 ml micro-centrifuge tube (Intermountain Scientific # 3260-1) incubated at room temperature for 5 minutes prior to the addition of 200 ul of chloroform. The upper phase is separated to a fresh 2 ml micro-centrifuge tube and diluted with an equal volume of 70% ethanol. The sample was then applied to an Rneasy column ( Qiagen#74104) for further extraction as per the manufacturers protocol. RNA is eluted in DEPC treated water to a final volume of 50 μl Samples are subsequently treated with Dnase (Promega # M610A) for 40 minutes at 37 degrees in a volume of 200 μl of DEPC water containing 1 mm DTT, 10 mm MgCl₂,10 mm Tris and 2 units of Dnase. RNA is extracted by adding an equal volume of a 1:1 phenol/chloroform mixture, separation of the upper phase by centrifugation (repeated xl) followed by a single chloroform extraction and precipitation with 2.5× volume of 100% ethanol and 1/10 volume of 5M NaCl at −20 degrees for 1-2 hours. The precipitated RNA is pelleted, air-dried for 20-30 minutes and re-dissolved in 30-50 μl of DEPC water. The yield of RNA is determined spectrophotometrically at 260 nm. Agarose gel electrophoresis confirms the RNA to contain both 28s and 18s species of RNA with little or no evidence of degradation. PCR amplification of the RNA template with Taq Polymerase confirms no DNA contamination. Similar methods have been utilized extracted mRNA using Pharmacia kits (see FIG. 12).

DNA is isolated from the remainder of the urea lysis buffer homogenate by a series of phenol/chloroform (500 μlx2) extractions using a 1:1 phenol/chloroform mix followed by a single chloroform (500 μl) extraction. DNA is precipitated at −20 degrees from the final extract by the addition of 25 μl of 5M NaCl and 850 μl of 100% ethanol following a minimum incubation time of 30 minutes. The DNA is pelleted by spinning at maximun speed for 15 minutes, washed once in 70% ethanol and air dried for 30-40 minutes. The DNA pellet is redissolved by repeated pipetting to a volume of 30-50 μl in water followed by incubation at 65 degrees for approximately 60 minutes to ensure complete solubilization of the sample. DNA yield is determined spectrophotometrically.

### Determination of % Recovery ability to trace loss

In order to assess any losses in RNA or DNA during the isolation procedure a series of experiments were conducted in which HIV negative control biopsies were “spiked” with increasing copy numbers of a known RNA (HIV LTR) and DNA (firefly luciferase) sequence prior to homogenization. The recovery of these sequences in the final RNA and DNA was determined by PCR using the relevant oligonucleotide primer pairs and expressed as a percentage of the copy numbers added prior to extraction. A series of experiments were conducted in which the external control sequences were added in increasing quantity in order to demonstrate recovery efficiency at different starting concentrations. Since the simultaneous isolation process involves the splitting of the initial sample homogenate, recovery experiments were also undertaken in which the sample was either divided equally for RNA and DNA extraction or else was processed without division. Recovery experiments demonstrated that both RNA and DNA can be extracted with high efficiency (83-100%) and that division of the homogenate does not favor the isolation one form of nucleic acid over the other (potential problem if the sample was not homogeneous). These techniques are now being modified to enable detection of loss and subsequent correction during handling/shipping by the use of prepackaged vials from the inventor's laboratory.

### PCR Quantitation: HIV RNA

Since tissue may contain many potentially inhibitory molecules, we performed a series of experiments in which a known copy number (previously ascertained to be within the linear amplification range) of Armored cyclophilin RNA (Ambion) were added to increasing amounts of tissue RNA and subjected to RT-PCR using the Thermo-stable rTth Reverse Transcriptase RNA PCR kit (Perkin-Elmer). The downstream cyclophilin primer is end labeled with P-32; radio-labeled PCR products are visualized and quantitated following polyacylamide gel electrophoresis exposure to a phospho-image plate and analysed using Image-Quant software (Molecular Dynamics). Such experiments demonstated that RNA concentrations of up to 261 ng/μl did not affect the amplification of the external control (>100%) with only a slight diminution in efficiency at concentrations up to 561 ng/ul (95%). An upper limit of 250 ng of RNA is added to the RT-PCR to avoid any potential inhibitory interactions.

HIV RNA is quantitated using the Thermostable rTth Reverse Transcriptase RNA PCR kit (Perkin-Elmer) in conjunction with the oligonucleotide primer pair 667/AA55 specific for the R/U5 region of the LTR (detects both spliced and unspliced HIV RNA). 667 was end-labeled with P-32 using T4-Kinase (Gibco-Life Technologies), was subjected to 30 cycles of PCR (94 deg/1 min 65 deg/2 mins) followed by an 8 minute elongation at 72 degrees along with a previously established linear standard curve prepared from a stock of purified HIV LTR RNA. PCR products were subjected to polyacrylamide gel electrophoresis and the radio-label incorporated in the specific 140bp LTR product is determined by phosphoimage analysis. Samples are quantitated with reference to the linear portion of the standard curve by importing the raw data into an excel spreadsheet. HIV RNA copies were expressed per jig of tissue RNA. Samples can also be expressed as a calculated result (number of copies per 106 cells) by back-calculation to determine the number of cells from which the RNA was derived. As DNA and RNA are extracted from the same sample (and same total sample volume) and equally divided with internal tracer controls (e.g. luciferase DNA and cyclophilin RNA), the amount of cells in the DNA sample can be determined using the housekeeping gene β-globin, corrected for loss (via luciferase tracking); the number of cells supporting the RNA assay can then be indirectly inferred and corrected for RNA loss.

### PCR Quantitation: HIV DNA

1-2 μg of DNA is amplified using the oligonucleotide primer pair AA55/667 recognizing the R/U5 region of the LTR and Taq Polymerase. As an internal standard 100-200 ng of DNA is amplified using oligonucleotide primers LA1 and LA2 specific for a 110 bp product of the β-globin gene. Both reactions are subjected to 30 cycles of PCR (94 deg/1 min 65 deg/2 mins) along with DNA standard curves for both products that are within a previously established linear amplification range. By end labeling the upstream oligonucleotide primer with P-32, radio-labeled product is resolved and quantitated by phospho-image analysis. Samples are quantitated with reference to the linear portion of the respective standard curves using an excel spreadsheet. HIV DNA copies are expressed per 2×10⁶ copies of β-globin.

### CCR5 co-receptor expression on mucosal CD4 T cells.

Isolation of mucosal mononuclear cells does not alter phenotypic expression of CD4, CD8, CCR5 and CXCR4.

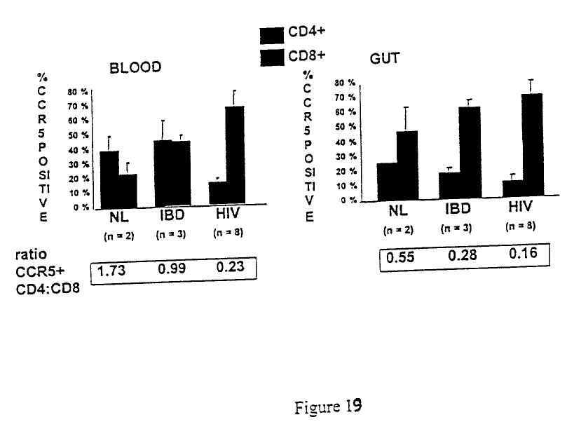

Samples were isolated from healthy, seronegative controls' blood (peripheral blood mononuclear cells: PBMC) and intestinal mucosa (mucosal mononuclear cells: MMC) to establish baseline CCR5 and CXCR4 expression in both compartments. FIG. 19 demonstrates that our isolation procedure neither strips relevant receptors (CD4, CD8, CCR5, CXCR4) nor alters their surface expression.

Mucosal expression of CCR5 on CD4 T cells is greatly increased compared with PBMC. Isolated mucosal mononuclear cells and peripheral blood mononuclear cells were obtained from healthy, sero-negative control subjects and evaluated to determine the relative percentages of CD4 T lymphocytes in each compartment expressing CCR5 receptors. The 2D7 CCR5 antibody is conjugated in a 1:1 ratio with phycoerythrin; the flow cytometry instruments used are calibrated to detect 44 phycoerythrin molecules per RFI channel (based on a standardized CD4 expression and number of antibodies bound per cell). Consequently, the number of anti-CCR5 antibodies bound per cell can be translated to number of receptors per cell, assuming monovalent binding of antibody to receptor.

The percentages of CCR5-expressing CD4+ T cells is significantly increased in the gut (87%) compared to the blood (11%) (p=0.0019).

Further enhancing the vulnerability to HIV infection, shows mucosal CD4 T cells also express significantly more receptors per cell (mean of 8500) compared to blood CD4 T cells (mean 2700)(p=0.007).

CCR5 expression on mucosal CD4 T cells is nearly exclusively on the memory subset. Blood and mucosal samples from the same seronegative, healthy controls were counter stained with CD45RO antibody as an indicator of the memory subset to determine the relative distribution of CCR5 staining. After gating on CD4+ fluorescence, 91% of CD4+ CD45RO+mucosal cells express CCR5 compared to 24% of a matched group in the blood (p=0.017).

### CCR5 expression on mucosal CD4 T cells remains increased compared to PBMC in HIV-infected and inflammatory samples.

Having ascertained preliminary baselines of CCR5 expression in mucosal and blood CD4 T cells in healthy, seronegative subjects, the expression on CD4 T cells in the setting of chronic HIV infection was evaluated to test the hypothesis that CCR5 expression would remain increased in the mucosa compared to blood, favoring HIV replication. Inflammatory controls were included to discern changes not directly related to HIV-infection. Inflammatory controls were well-characterized subjects with inflammatory bowel disease (IBD), specifically, ulcerative colitis. Subjects were clinically in remission (maintained but controlled mucosal inflammation) on 5-ASA anti-inflammatory agents only, no steroids or immunosuppressive medications were used. HIV-infected individuals had peripheral CD4 counts between 200-700 cells/mm3 with a range of plasma viral loads (undetectable by ultrasensitive assay: n=2; plasma viral load between 200-2000 copies/ml: n=2; plasma viral load between 20,000-40,000 copies/ml: n=4).

The differential expression of CCR5 between mucosal and blood CD4 T cells observed in seronegative normal controls was maintained in inflammatory controls (p=0.012) and HIV-infected subjects (p=0.04)(FIG. 18). In agreement with our hypothesis, there is a trend toward significance identifying a decrease in mucosal CCR5 expression on CD4 T cells in HIV compared with normal controls as shown in this figure.

### The CD4:CD8ratio of CCR5-expressing T cells decreases in HIV and IBD in both blood and gut.

CCR5 receptor on CD4 T cells are also expressed on CD8+ T cells. To further evaluate if there was a true decrease in CCR5 expression on mucosal CD4 T cells, evaluation of the relative distribution of CCR5 receptors between CD4 and CD8T lymphocytes in both compartments in the three clinical conditions. FIG. 19 demonstrates the dramatic downward shifts in the CD4:CD8 ratios of CCR5 expressing cells, decreasing in blood and gut samples by roughly 50% in IBD and 70-90% in HIV. This may represent a protective down regulation of CCR5 to inhibit HIV spread. Given the similar trend in the inflammatory controls, the primary stimulus for decreased surface expression likely relates to the inflammatory milieu. Samples are being processed to confirm that b-chemokine levels are elevated in both conditions, but even more so in HIV than in IBD samples. Supporting our hypothesis, these findings would suggest an extremely active inflammatory mucosal state in HIV (as defined by chemokine activity) despite the histological reports of relative lymphopenia.

Mucosal mononuclear cells (MMC) are significantly more infectible in vitro than PBMC. Initial observations demonstrated increased vulnerability to HIV infection of mucosal cells due, in part, to increased co-receptor expression. This hypothesis was tested in vitro using isolated MMC and PBMC from the same individuals and incubated with M-tropic HIVSX for 2 hours, washed and cultured for 3-10 days. Aliquots of supernatant were collected at the demonstrated times and assayed fro p24 production as evidence of infection. The p24production at the first time point (3 days) was markedly increased compared to concurrently incubated PBMC (4000 ng p24/ml in MMC compared to 550 ng p24/ml in PBMC). These pilot data support our hypothesis that the increased co-receptor expression on mucosal CD4 T cells enhances infectivity by HIV.

A number of embodiments of the present invention have been described. Nevertheless, it will be understood that various modifications may be made without departing from the spirit and scope of the invention.

What is claimed is:

1. A method of determining a specific nucleic acid sequence in a non-fluid biological sample, comprising obtaining a biolgical sample from the subject and quantifying the amount of the specific nucleic acid present in the sample.

2. The method of claim 1, wherein the specific nucleic acid is related to a pathologic condition.

3. The method of claim 1, wherein the specific nucleic acid is a bacterial nucleic acid.

4. The method of claim 1, wherein the specific nucleic acid is a vial nucleic acid.

5. The method of claim 1, wherein the sample is from a mammal.

6. The method of claim 5, wherein the mammal is a human.

7. The method of claim 1, wherein the non-fluid biological sample is selected from the group consisting of a cellular sample and a tissue sample.

8. The method of claim 7, wherein the tissue sample is selected from the group consisting of mucosal tissue, skin tissue, lymph tissue, ocular tissue, pulmonary tissue, and liver tissue.

9. The method of claim 8, wherein the mucosal tissue sample is selected from the group consisting of gastro-intestinal tissue, uro-genital tissue , and nasal-larynx tissue.

10. The method of claim 1, wherein the tissue is a gut-associated lymphoid tissue.

11. The method of claim 1, wherein the quantification is by RT-PCR or direct PCR.

12. The method of claim 1, wherein the qualification is by northern or southern blots.

13. The method of claim 1, wherein the quantification is by RNAse protection assay.

14. The method of claim 1, wherein the quantification is by detection of a viral polypeptide.

15. The method of claim 1, further comprising spiking the sample with a known quantity of a nucleic acid.

16. A method for determining the effect of a therapeutic treatment on whole body viral load in a subject, comprising determining the viral load in the plasma of the subject and the viral load in a tissue-biopsy of the subject compared to a standard whole body viral load, wherein a change in the viral load in the blood and the tissue is indicative of an effect.

17. The method of claim 16, wherein the subject is a mammal.

18. The method of claim 17, wherein the mammal is a human.

19. The method of claim 17, wherein the tissue sample is a mucosal tissue sample.

20. The method of claim 17, wherein the mucosal tissue sample is selected from the group consisting of gastro-intestinal tissue, uro-genital tissue , and nasal-larynx tissue.

21. The method of claim 17, wherein the tissue is a gut-associated lymphoid tissue.

22. The method of claim 17, wherein the tissue is liver or skin.

23. The method of claim 17, wherein the quantification is by RT-PCR.

24. The method of claim 17, wherein the qualification is by northern or southern blots.

25. The method of claim 17, wherein the quantification is by RNAse protection assay.

26. The method of claim 17, wherein the quantification is by detection of a viral polypeptide.

27. The method of claim 17, wherein the quantification is by detecting a nucleic acid.

28. The method of claim 22, wherein the nucleic acid is DNA or RNA.

29. A kit for obtaining a non-fluid biological sample from the skin, the kit comprising:
a collection device;
a known quantity of a nucleic acid; and
a cell lysis buffer suitable of preserving nucleic acids.
